(19) United States
(12) Patent Application Publication
(10) Pub. No.: US 2012/0283493 A1
(43) Pub. Date: Nov. 8, 2012

(54) MULTIPRODUCT BIOREFINERY FOR SYNTHESIS OF FUEL COMPONENTS AND CHEMICALS FROM LIGNOCELLULOSICS VIA LEVULINATE CONDENSATIONS

(76) Inventors: Edwin S. Olson, Grand Forks, ND (US); Carsten Heide, Grand Forks, ND (US)

(21) Appl. No.: 13/413,881

(22) Filed: Mar. 7, 2012

Related U.S. Application Data
(63) Continuation-in-part of application No. 12/795,479, filed on Jun. 7, 2010.
(60) Provisional application No. 61/184,456, filed on Jun. 5, 2009.

(51) Int. Cl. C07C 1/22

(52) U.S. Cl. 585/242; 585/331; 585/317; 585/240; 585/250

(57) ABSTRACT

An integrated method for production of fuels, fuel additives, or chemicals in a biorefinery by the conversion of cellulosic materials is disclosed herein. The method is based on converting a source of C6 sugar into a mixture of hydrotreated compounds. Embodiments of the method can be highly integrated, with reagents for particular steps being provided by other steps of the process.

### CROSS-REFERENCE TO RELATED APPLICATIONS

This application is a continuation-in-part of and claims the benefit of priority under 35 U.S.C. §120 to U.S. Utility application Ser. No. 12/795,479 entitled “Multiproduct Biorefinery for Synthesis of Fuel Components and Chemicals from Lignocellulosics via Levulinate Condensations,” filed Jun. 7, 2010, which claims the benefit under 35 U.S.C. §119(e) of U.S. Provisional Application No. 61/184,456, filed Jun. 5, 2009, the disclosures of which are hereby incorporated herein by reference.

### STATEMENT REGARDING FEDERALLY SPONSORED RESEARCH OR DEVELOPMENT

This invention was made with government support under the Cooperative Agreement No. DE-FG36-08GO88054 entitled “EERC Center for Biomass Utilization 2009,” awarded by the U.S. Department of Energy. The government has certain rights in the invention.

### FIELD OF THE INVENTION

This invention relates to a process for production of liquid fuels, fuel additives, or chemicals by the conversion of cellulosic materials.

### BACKGROUND

Although cellulose is the most abundant plant material resource, its exploitation has been curtailed by its composite nature and rigid structure. As a result, most technical approaches to convert lignocellulosic material to fuel products have focused on an effective pretreatment to liberate the cellulose from the lignin composite and break down its rigid structure. Besides effective cellulose liberation, a favorable pretreatment can minimize the formation of degradation products because of their wastefulness and inhibitory effects on subsequent processes. One way to improve the efficiency of biomass conversion schemes (biorefineries) is to integrate the energy-intensive lignocellulose depolymerization and dehydration (LDD) process with power production and/or other biomass processing. Some biorefineries rely on conversion of lignocellulose to glucose and subsequent fermentation, but this processing can require expensive enzymes and long contact times or can produce compounds that inhibit the fermentation or that are low-value by-products. In addition, fermentation releases carbon dioxide and produces cell mass, which in some examples can only be efficiently reused as a livestock supplement.

An alternative processing for lignocellulosic materials is acid-catalyzed depolymerization and conversion to the C5 product, levulinic acid, or esters thereof. In general, two methods are used to produce levulinic acid or levulinate ester from lignocellulose. One method uses water with a strong acid catalyst, such as sulfuric acid, to effect the depolymerization and dehydration of lignocellulose to produce the C5 and C1 acids (levulinic and formic acids) (see, for example, U.S. Pat. No. 5,608,105). However, separation of products from the aqueous product solution is difficult. One patent describes a separation scheme that uses an olefin feed to convert the aqueous acid to esters that can be separated from the water and each other (see, for example, U.S. Pat. No. 7,153,996). Of course, a nearby olefin source is required for this process.

Another method uses an alcohol solvent for the acid-catalyzed depolymerization of cellulose, which results in direct formation of the levulinate ester (see, for example, DE 3621517).

A recent U.S. Department of Energy-sponsored project at the Energy & Environmental Research Center showed that high yields of methyl and ethyl levulinates along with charcoal and resins are obtained from several agricultural and wood (particleboard) wastes using relatively easy purification procedures, with little wastewater production. Valuable furfural and alkyl formates were also formed in addition to recovered resin from the particleboard and charcoal.

Valerie biofuels have been proposed by hydrogenation of γ-valerolactone to valeric acid, ethyl valerate, butyl valerate, and pentyl valerate (Lange, J.-P.; Price, R.; Ayoub, P. M.; Louis, J.; Petrus, L.; Clarke, L; Gosslink, H. Angew. Chem. 2010, 49, 4479). The valeric platform potentially offers biofuels that can be used as components in both gasoline and diesel for blending. Nevertheless their acceptance as transportation fuels is challenged as they do not readily integrate in the existing petroleum fuel supply infrastructure.

The potential of levulinic acid and γ-valerolactone for biofuel manufacture has been also addressed by another method which converts γ-valerolactone into butenes via decarboxylation (see, for example, Science 2010, 327, 1110-1114). The ‘butenes can provide a feedstock for gasoline but not for diesel or jet fuel unless they are further oligomerized. However, this process may not be economically attractive.

### BRIEF SUMMARY OF SOME OF THE PREFERRED EMBODIMENTS

More efficient means for conversions of agricultural, forest, aquaculture algae, and construction waste to fuels and chemicals are sought so that useful biomass-derived products can compete with and be integrated with the production of petroleum-based products. A simple integrated method is needed to synthesize diesel and jet fuels, diesel additives, solvents, and plasticizers from C5 intermediates, levulinic acid, or levulinic acid esters with appropriate reagents, such that product streams are readily separable and such that useful higher molecular weight compounds are provided.

In various embodiment, the present invention provides a method for converting a source of C6 sugar into a mixture of hydrotreated compounds. The method includes thermocatalytically reacting a source of C6 sugar to produce a solution. The solution includes at least one of levulinic acid and levulinic acid ester. The method includes extracting at least one of the levulinic acid and the levulinic acid ester from the solution. The extraction is performed using a cyclic ether. The method also includes condensing at least a portion of at least one of the levulinic acid and the levulinic acid ester with at least one of C4-C1 aldehydes, C4-C11 ketones, C4-C11 esters, or C4-C11 ketoacids to produce a condensation product. The method also includes hydrotreating at least a portion of the condensation product to provide a mixture of hydrotreated compounds.

In various embodiments, this invention includes a set of integrated processes for achieving the desired goal of fuel and chemical production in a biorefinery. The biorefinery can operate in a unique parallel processing mode wherein the initial biomass feedstocks are initially broken into smaller chemical components to provide substrates for parallel branches of the process, whose products can be allowed to react, for example, in a condensation step or a mixed hydrotreating step, as illustrated herein. The product streams of the biorefinery can include longer molecular weight products with a carbon chain length of about 8 or higher created from the condensation or hydrotreating step and shorter molecular weight by-products from unreacted starting materials.

Processing of the lignocellulosics can include their conversion to levulinic acid or esters thereof, which can condense with other compounds (which may or may not be derived from the lignocellulosic feedstock), which can be hydrotreated to produce fuels or fuel components. The fuel or blended fuel components can form a composition with the appropriate combustion properties and other physical properties for the desired fuel type.

Various embodiments of the present invention can provide certain advantages over other methods of converting lignocellulosic materials into other valuable materials.

Other biomass-to-fuel process schemes have little or no control over the molecular weight range of the fuel products, often producing high molecular weight materials that require subsequent cracking reactions to attain the desired range of carbon chains. This often occurs during the direct hydrotreating of carbohydrate materials and can result in plugging the reactors. Various embodiments of the present invention advantageously employ a concept of controlled breakdown, controlled condensation, and proper combining of the parallel streams in the condensation or hydrotreating steps, allowing the hydrotreating reactors to achieve the desired fuel blends.

For example, one advantageous aspect of this invention is focused on the alternative catalytic processing of lignocellulose that directly produces good yields of a mixture of C5 and C1 acids or esters thereof accompanied by compounds including valuable furfural. The catalytic processing of cellulosic biomass in alcohols offers a direct conversion to levulinate and formate esters that are useful for fuels and chemical intermediates. Levulinic acid and esters thereof are considered potential platform chemicals. The alkyl levulinates can be valuable intermediates for formation of plasticizers. In various embodiments, an acid-catalyzed depolymerization conducted in acidic ethanol results in formation of ethyl levulinate that is more easily extracted than levulinic acid from the corresponding aqueous reaction. The higher solubility in organic solvents also facilitates processing the condensation products and introduction into the hydrotreating reactor.

In another example, another advantageous aspect of this invention can be integration of a pyrolysis pretreatment step of cellulosic biomass. The biomass can be depolymerized in such a thermal unit to give a soluble carbohydrate intermediate, such as anhydrosugars, prior to conversion to levulinic acid or esters thereof. In the thermocatalytic reaction, the anhydrosugars can be directly converted into ethyl levulinate or reagent aldehydes for the condensation step. Generally anhydrosugars from pyrolysis cannot be fermented without extensive purification and transformation which result in loss of materials and large expenses. They are also difficult to hydrotreat at high temperatures to convert directly to fuel hydrocarbons. In various embodiments, the conversion of anhydrosugars to ethyl levulinate in an ethanol solution over a solid acid catalyst represents an advantageous high yield preconversion step for the process.

Another advantageous aspect of various embodiments of the invention is, for example, to convert the C5 acids or esters thereof into fuel blendstocks for the production of finished fuels that meet petroleum-based fuel specifications. Some embodiments of the present invention can achieve this goal by integrating production of the levulinic acid derivatives and the levulinic acid ester derivatives with the processing of other portions of feedstock via condensation of appropriate intermediates, which can result in a range of further intermediates with desired carbon chain lengths for fuels.

In another example, an advantageous aspect of this invention is the integration of the reduction of fatty acid derivatives from the thermocatalytically treated feedstocks with reduction of the condensation products to produce fuel blendstocks including paraffins, isoparaffins, cycloparaffins, and alkylaromatics, which can allow satisfaction of physical property specifications for jet fuels, such as Jet-A or JetA1, for example.

Various embodiments advantageously allow production of cyclic ethers via mild hydrotreating of the condensation products. These cyclic ethers can be utilized as diesel fuel additives to boost cetane value and reduce particulate emissions from the diesel combustion process. In some embodiments, this method is further integrated and uses light cyclic ethers, such as methyl tetrahydrofuran, which can occur as by-products, as a solvent for the isolation of the levulinic acid or the levulinic acid ester products from the depolymerization reaction. In some examples, cyclic ether byproducts can be used as a blendstock for gasoline.

In some embodiments, this method advantageously integrates the catalytic processing of lignocellulosic materials in order to meet some types of jet fuel specifications, a fuel can include some of each of the types of hydrocarbons described above, as well as an appropriate distribution of carbon chain lengths. Blending of the streams from the parallel processing biorefinery can allow an integrated process.

### BRIEF DESCRIPTION OF THE DRAWINGS

In the drawings, which are not necessarily drawn to scale, like numerals describe substantially similar components throughout the several views. Like numerals having different letter suffixes represent different instances of substantially similar components. The drawings illustrate generally, by way of example, but not by way of limitation, various embodiments discussed in the present document.

FIG. 1 is a schematic of an integrated C5 biorefinery for oil seed biomass conversion to fuels via levulinate and isobutyraldehyde, in accordance with various embodiments.

FIG. 2 is a schematic of an integrated C5 biorefinery for lignocellulose conversion to fuels via ethoxymethyfurfural or furfural, in accordance with various embodiments.

FIG. 3 is a schematic of an integrated C5 biorefinery employing the products and by-products for conversion to fuels, in accordance with various embodiments.

FIG. 4 is a schematic of an integrated C5 biorefinery fruit and sugar beet wastes and a solid acid conversion unit for the soluble portion, in accordance with various embodiments.

FIG. 5 is a schematic of an integrated C5 biorefinery for algae biomass conversion to fuels via ethyl levulinate and ethoxymethyl furfural or furfural, in accordance with various embodiments.

FIG. 6 is a schematic of an integrated C5 biorefinery for lignocellulose conversion to fuels via anhydrosugars and levulinate, in accordance with various embodiments.

FIG. 7 is a schematic of the depolymerization/decomposition of cellulose in ethanol and sulfuric acid, followed by a condensation reaction of ethyl levulinate with an aldehyde, in accordance with various embodiments.

FIG. 8 is a schematic of a condensation product with furfural and subsequent Diels-Alder reaction and reduction to cycloparaffin, in accordance with various embodiments.

FIG. 9 is a schematic of hydrogenation of levulinate intermediates, in accordance with various embodiments:

A. severe hydrogenation to alkanes,

B. hydroisomerization to isoparaffins, and

C. mild hydrogenation to alkyl tetrahydrofurans.

FIG. 10 is a schematic for the extraction and purification of the product mixture in unit (150) from reactor (100), in accordance with various embodiments.

FIG. 11 illustrates a GC-MS chromatogram of product collected, in accordance with various embodiments.

FIG. 12 illustrates conversion data for condensate hydrogenation, in accordance with various embodiments.

FIG. 13 illustrates a GC-MS chromatogram after hydrotreating over a Cu/Pd/carbon catalyst, in accordance with various embodiments.

FIG. 14 illustrates a GC-MS chromatogram of the hydrotreated product from Feed 1 with Ni—Mo catalyst, in accordance with various embodiments.

FIG. 15 illustrates a GC-MS chromatogram of the hydrotreated product from Feed 2 with Ni—Mo Catalyst, in accordance with various embodiments.

FIG. 16 illustrates a GC-MS chromatogram of jet range center distillation cut, in accordance with various embodiments.

FIG. 17 illustrates a schematic of an integrated biorefinery for the conversion of sources of C5 and C6 sugars to fuels and cyclic ethers via alcoholysis, in accordance with various embodiments.

FIG. 18 illustrates is a schematic of an integrated biorefinery for the conversion of sources of C5 and C6 sugars to fuels, levulinic acid, and cyclic ethers via hydrolysis, in accordance with various embodiments.

### DETAILED DESCRIPTION

Reference will now be made in detail to certain claims of the disclosed subject matter, examples of which are illustrated in the accompanying drawings. While the disclosed subject matter will be described in conjunction with the enumerated claims, it will be understood that they are not intended to limit the disclosed subject matter to those claims. On the contrary, the disclosed subject matter is intended to cover all alternatives, modifications, and equivalents, which can be included within the scope of the presently disclosed subject matter as defined by the claims.

References in the specification to “one embodiment,” “an embodiment,” “an example embodiment,” etc., indicate that the embodiment described can include a particular feature, structure, or characteristic, but every embodiment may not necessarily include the particular feature, structure, or characteristic. Moreover, such phrases are not necessarily referring to the same embodiment. Further, when a particular feature, structure, or characteristic is described in connection with an embodiment, it is submitted that it is within the knowledge of one skilled in the art to affect such feature, structure, or characteristic in connection with other embodiments whether or not explicitly described.

Values expressed in a range format should be interpreted in a flexible manner to include not only the numerical values explicitly recited as the limits of the range, but also to include all the individual numerical values or sub-ranges encompassed within that range as if each numerical value and sub-range is explicitly recited. For example, a concentration range of “about 0.1% to about 5%” should be interpreted to include not only the explicitly recited concentration of about 0.1 wt % to about 5 wt %, but also the individual concentrations e.g., 1%, 2%, 3%, and 4%) and the sub-ranges e.g., 0.1% to 0.5%, 1.1% to 2.2%, 3.3% to 4.4%) within the indicated range.

in this document, the terms “a,” “an,” or “the” are used to include one or more than one unless the context clearly dictates otherwise. The term “or” is used to refer to a nonexclusive “or” unless otherwise indicated. In addition, it is to be understood that the phraseology or terminology employed herein, and not otherwise defined, is for the purpose of description only and not of limitation. Any use of section headings is intended to aid reading of the document and is not to be interpreted as limiting; information that is relevant to a section heading may occur within or outside of that particular section. Furthermore, all publications, patents, and patent documents referred to in this document are incorporated by reference herein in their entirety, as though individually incorporated by reference. In the event of inconsistent usages between this document and those documents so incorporated by reference, the usage in the incorporated reference should be considered supplementary to that of this document; for irreconcilable inconsistencies, the usage in this document controls.

In the methods of manufacturing described herein, the steps can be carried out in any order without departing from the principles of the invention, except when a temporal or operational sequence is explicitly recited.

Furthermore, specified steps can be carried out concurrently unless explicit claim language recites that they be carried out separately. For example, a claimed step of doing X and a claimed step of doing Y can be conducted simultaneously within a single operation, and the resulting process will fall within the literal scope of the claimed process.

R-groups designate any suitable substituent, such as H or any suitable organic group, or any other suitable substituent, unless otherwise specified herein,

### Definitions

The term “about” can allow for a degree of variability in a value or range, for example, within 10%, within 5%, or within 1% of a stated value or of a stated limit of a range. When a range or a list of sequential values is given, unless otherwise specified any value within the range or any value between the given sequential values is also disclosed.

The term “alkyl” as used herein refers to straight chain and branched alkyl groups and cycloalkyl groups having from 1 to 40 carbon atoms, 1 to about 20 carbon atoms, from 1 to 12 carbons or, in some embodiments, from 1 to 8 carbon atoms. Examples of straight chain alkyl groups include those with from 1 to 8 carbon atoms such as methyl, ethyl, n-propyl, n-butyl, n-pentyl, n-hexyl, n-heptyl, and n-octyl groups. Examples of branched alkyl groups include, but are not limited to, isopropyl, iso-butyl, sec-butyl, t-butyl, neopentyl, isopentyl, and 2,2-dimethylpropyl groups. As used herein, the term “alkyl” encompasses n-alkyl, isoalkyl, and anteisoalkyl groups as well as other branched chain forms of alkyl. Representative substituted alkyl groups can be substituted one or more times with any of the groups listed herein, for example, hydroxy, carboxy, nitro, and alkoxy groups.

The term “alkenyl” as used herein refers to straight and branched chain and cyclic alkyl groups as defined herein, except that at least one double bond exists between two carbon atoms. Thus, alkenyl groups have from 2 to about 40 carbon atoms, 2 to about 20 carbon atoms, or 2 to 12 carbons or, in some embodiments, from 2 to 8 carbon atoms. Examples include, but are not limited to vinyl, —CH═CH(CH₃), —CH═C(CH₃)₂, —C(CH₃)═CH₂, —C(CH₃)═CH(CH₃), —C(CH₂CH₃)═CH₂, cyclohexenyl, cyclopentenyl, cyclohexadienyl, butadienyl, pentadienyl, and hexadienyl among others.

The term “acyl” as used herein refers to a group containing a carbonyl moiety wherein the group is bonded via the carbonyl carbon atom. The carbonyl carbon atom is also bonded to another carbon atom, which can be part of an alkyl, aryl, aralkyl cycloalkyl, cycloalkylalkyl, heterocyclyl, heterocyclylalkyl, heteroaryl, heteroarylalkyl group or the like. In the special case wherein the carbonyl carbon atom is bonded to a hydrogen, the group is a “formyl” group, an acyl group as the term is defined herein. An acyl group can include 0 to about 12-20 additional carbon atoms bonded to the carbonyl group. An acyl group can include double or triple bonds within the meaning herein. An acryloyl group is an example of an acyl group. An acyl group can also include heteroatoms within the meaning here. A nicotinoyl group (pyridyl-3-carbonyl) group is an example of an acyl group within the meaning herein.

The term “cycloalkyl” as used herein refers to cyclic alkyl groups such as, but not limited to, cyclopropyl, cyclobutyl, cyclopentyl, cyclohexyl, cycloheptyl, and cyclooctyl groups. In some embodiments, the cycloalkyl group can have 3 to about 8-12 ring members, whereas in other embodiments the number of ring carbon atoms range from 3 to 4, 5, 6, or 7. Cycloalkyl groups further include polycyclic cycloalkyl groups such as, but not limited to, norbornyl, bornyl, camphenyl, isocamphenyl, and carenyl groups, and fused rings. Cycloalkyl groups also include rings that are substituted with straight or branched chain alkyl groups as defined herein. The term “cycloalkenyl” alone or in combination denotes a cyclic alkenyl group.

The term “aryl” as used herein refers to cyclic aromatic hydrocarbons that do not contain heteroatoms in the ring. Thus aryl groups include, but are not limited to, phenyl and naphthyl groups. In some embodiments, aryl groups contain about 6 to about 14 carbons in the ring portions of the groups. Aryl groups can be unsubstituted or substituted, as defined herein. Representative substituted aryl groups can be mono-substituted or substituted more than once. The term “aralkyl” as used herein refers to alkyl groups as defined herein in which a hydrogen or carbon bond of an alkyl group is replaced with a bond to an aryl group as defined herein. Representative aralkyl groups include benzyl and phenylethyl groups and fused (cycloalkylaryl)alkyl groups such as 4-ethyl-indanyl. Aralkenyl group are alkenyl groups as defined herein in which a hydrogen or carbon bond of an alkyl group is replaced with a bond to an aryl group as defined herein.

The term “heterocyclyl” as used herein refers to aromatic and non-aromatic ring compounds containing 3 or more ring members, of which, one or more is a heteroatom such as, but not limited to, N, O, and S. Thus a heterocyclyl can be a cycloheteroalkyl, or a heteroaryl, or if polycyclic, any combination thereof. In some embodiments, heterocyclyl groups include 3 to about 20 ring members, whereas other such groups have 3 to about 15 ring members. A heterocyclyl group designated as a C₂-heterocyclyl can be a 5-ring with two carbon atoms and three heteroatoms, a 6-ring with two carbon atoms and four heteroatoms and so forth. Likewise a C₄-heterocyclyl can be a 5-ring with one heteroatom, a 6-ring with two heteroatoms, and so forth. The number of carbon atoms plus the number of heteroatoms sums up to equal the total number of ring atoms. A heterocyclyl ring can also include one or more double bonds. A heteroaryl ring is an embodiment of a heterocyclyl group. The phrase “heterocyclyl group” includes fused ring species including those that include fused aromatic and non-aromatic groups. For example, a dioxolanyl ring and a benzdioxolanyl ring system (methylenedioxyphenyl ring system) are both heterocyclyl groups within the meaning herein. The phrase also includes polycyclic ring systems containing a heteroatom such as, but not limited to, quinuclidyl. Heterocyclyl groups can be unsubstituted, or can be substituted as discussed herein. Heterocyclyl groups include, but are not limited to, pyrrolidinyl, piperidinyl, piperazinyl, morpholinyl, pyrrolyl, pyrazolyl, triazolyl, tetrazolyl, oxazolyl, isoxazolyl, thiazolyl, pyridinyl, thiophenyl, benzothiophenyl, benzofuranyl, dihydrobenzofuranyl, indolyl, dihydroindolyl, azaindolyl, indazolyl, benzimidazolyl, azahenzimidazolyl, benzoxazolyl, benzothiazolyl, benzothiadiazolyl, imidazopyridinyl, isoxazolopyridinyl, thianaphthalenyl, purinyl, xanthinyl, adeninyl, guaninyl, quinolinyl, isoquinolinyl, tetrahydroquinolinyl, quinoxalinyl, and quinazolinyl groups. Representative substituted heterocyclyl groups can be mono-substituted or substituted more than once, such as, but not limited to, piperidinyl or quinolinyl groups, which are 2-, 3-, 4-, 5-, or 6-substituted, or disubstituted with groups such as those listed herein.

The term “heteroaryl” as used herein refers to aromatic ring compounds containing 5 or more ring members, of which, one or more is a heteroatom such as, O; for instance, furanyl The term “heterocyclylalkyl” as used herein refers to alkyl groups as defined herein in which a hydrogen or carbon bond of an alkyl group as defined herein is replaced with a bond to a heterocyclyl group as defined herein. Representative heterocyclyl alkyl groups include, but are not limited to, furan-2-yl methyl, furan-3-yl methyl, pyridine-3-yl methyl, tetrahydrofuran-2-yl ethyl, and indol-2-yl propyl.

The term “heteroarylalkyl” as used herein refers to alkyl groups as defined herein in which a hydrogen or carbon bond of an alkyl group is replaced with a bond to a heteroaryl group as defined herein.

The term “alkoxy” as used herein refers to an oxygen atom connected to an alkyl group, including a cycloalkyl group, as are defined herein. Examples of linear alkoxy groups include but are not limited to methoxy, ethoxy, propoxy, butoxy, pentyloxy, hexyloxy, and the like. Examples of branched alkoxy include but are not limited to isopropoxy, sec-butoxy, tert-butoxy, isopentyloxy, isohexyloxy, and the like. Examples of cyclic alkoxy include but are not limited to cyclopropyloxy, cyclobutyloxy, cyclopentyloxy, cyclohexyloxy, and the like. An alkoxy group can include one to about 12-20 or about 12-40 carbon atoms bonded to the oxygen atom, and can further include double or triple bonds, and can also include heteroatoms. For example, an allyloxy group is an alkoxy group within the meaning herein. A methoxyethoxy group is also an alkoxy group within the meaning herein, as is a methylenedioxy group in a context where two adjacent atoms of a structures are substituted therewith.

The term “hydrocarbon” as used herein refers to a functional group or molecule that includes carbon and hydrogen atoms. The term can also refer to a functional group or molecule that normally includes both carbon and hydrogen atoms but wherein all the hydrogen atoms are substituted with other functional groups.

The term “source of C6 sugar” as used herein refers to cellulosic materials, starch materials, or C6 sugars. Examples of a source of C6 sugars can include suchrose, fructose, dextrose, molasses, raffinate, wood, wood pulp, pulping sludge, particleboard, paper, grass, agricultural by-product, straw, stalks, cobs, beets, beet pulp, seed hulls, bagasse, algae, corn starch, potato waste, sugar cane, for fruit waste.

The term “fuel” as used herein can refer to a hydrocarbon mixture, such as for example a distillate fuel, jet fuel, diesel fuel, compression ignition fuel, gasoline, spark ignition fuel, rocket fuel, marine fuel, or other fuel, qualifying as such by virtue of having a set of chemical and physical properties that comply with requirements delineated in a specification developed and published by ASTM (American Society of Testing and Materials), European Standards Organization (CEN), and/or the U.S. Military. In some examples, a fuel can be a liquid transportation fuel, for example, for surface or air transport. Surface transport includes both terra firma and oceanic transport. Fuels of this type are included, but not limited to, ASTM specifications D975 (Diesel Fuel Oil), D1655 (Aviation Turbine Fuels), D4814 (Automotive Spark Ignition Fuel); military specifications MIL-DTL-83133G (Turbine Fuel, Aviation, Kerosene Type), MIL-DTL-25576D (Propellant, Rocket Grade Kerosene), MIL-DTL-38219D (Turbine Fuel, Low Volatility), MIL-DTL-5624U (Turbine Fuel, Aviation), MIL-DTL-16884L (Fuel, Naval Distillate), and other such specifications for similar fuels.

The term “blendstock” as used herein refers to a composition that can be blended with any other suitable composition to form a fuel. A fuel additive can be a blendstock. A blendstock can form any suitable proportion of the final fuel product, for example about 1 wt %, 5 wt %, 10 wt %, 20 wt %, 30 wt %, 40 wt %, 50 wt %, 60 wt %, 70 wt %, 80 wt %, 90 wt %, 95 wt %, or about 99 wt % of the final product. In some examples, distillation can be used to form distinct blendstocks (e.g. having a particular range of hydrocarbon chain lengths or particular proportions of certain types of hydrocarbon compounds) from a product mixture, and any number of different distinct blendstocks forms from one or different products can be blended in suitable proportions to form a fuel.

### Description

### 1. General—Integrated Biorefinery

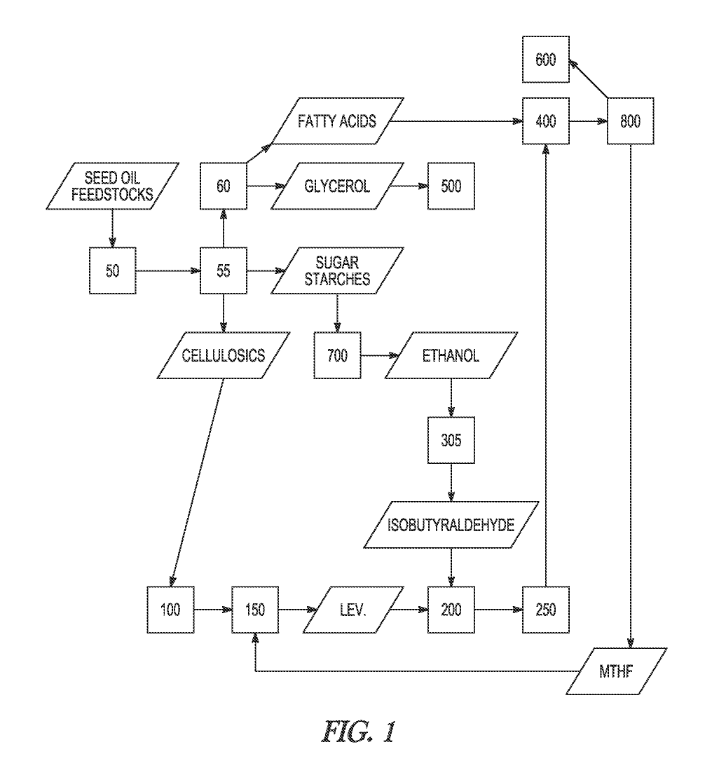

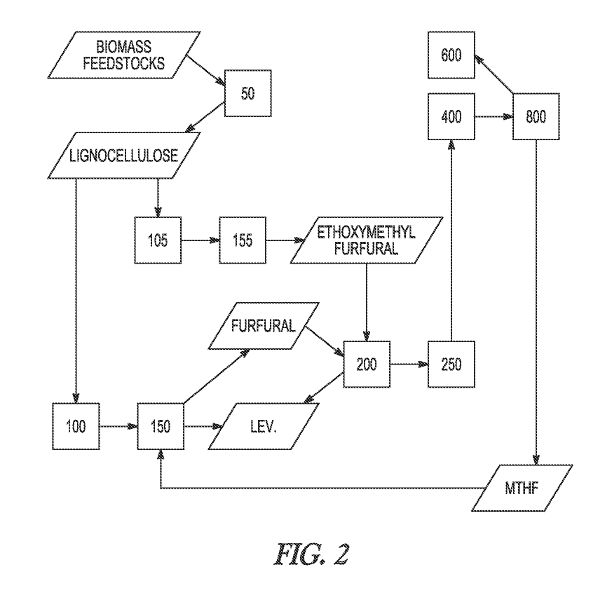

As illustrated in FIGS. 1 and 2, one of the preferred embodiments for the parallel processing C5 biorefinery is an integrated biorefinery including an initial separation (disassembly) unit (50 and or 55) for certain types of biomass containing oil where noncellulosic feedstocks are separated from cellulosic or lignocellulosic feedstocks, a cellulose depolymerization and dehydration (CDD, thermocatalytic treatment) unit (100) that catalytically depolymerizes and decomposes or reforms the lignocellulose; a condensation unit (200) that condenses the primary product from the thermocatalytic unit with a reactant aldehyde, ester, or ketone produced in a reagent production unit (300) from preferably renewable resources; and a hydrotreating unit (400) that converts the condensation products to fuels via hydrotreating. Additional units are added to convert by-products to chemical feedstocks and to separate and blend fuel components. A separation unit (150) can be added between the thermocatalytic unit (100) and the condensation unit (200) to recover the acid catalyst as well as to remove char and other by-products. In another embodiment of this invention, another separation unit (250) is added after the condensation unit (250) to recover unreacted product, recover catalysts, and remove water. In some embodiments, a separation unit (800) is added to recover the methyltetrahydrofuran (e.g. 2-methyltetrahydrofuran, also MTHF or MeTHF). In addition, with separation unit (800) cyclic ethers and tighter carbon compounds can be removed before running the product stream into a second hydrotreating unit (600) to produce high-quality hydrocarbon fuels and fuel blendstocks. Other energy crops, such as algae, can be processed similarly (FIG. 6).

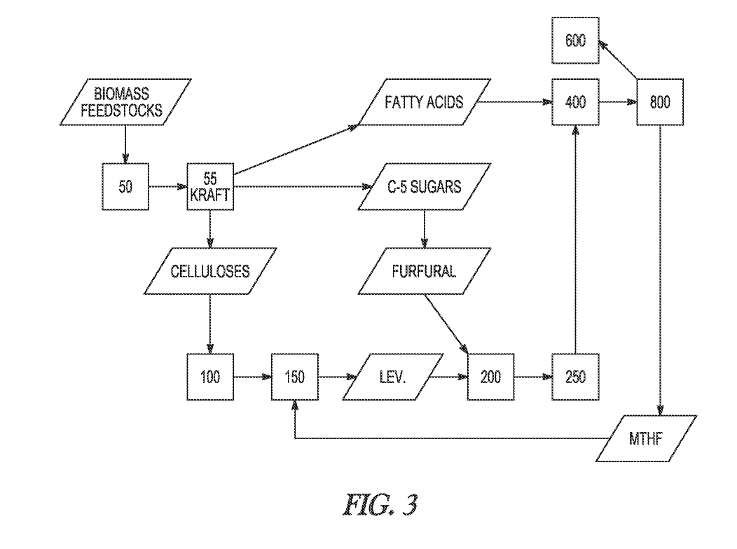

Alternatively, the process uses abundant cellulosic or lignocellulosic feedstocks (FIGS. 2, 3) including very low cost or negative cost wood and agriculture residue or grass and other energy crops. Lignocellulosic feedstocks are low in nitrogen and sulfur. The catalytic conversion to a levulinic acid or ester thereof can be highly efficient in producing a material appropriate for further chemical synthesis because of the chemical functionality retained in this conversion. In an embodiment of the invention, furfural can be condensed out in separation unit (150) and separated from the levulinic acid, as illustrated in FIG. 2.

Subsequent catalytic condensation reactions of the levulinic acid or levulinic acid ester in the condensation unit (200) permit its conversion to higher molecular weight species (see FIGS. 1-8). Thus the 5-carbon acyl group of the ester is combined with aldehydes or ketones to form (5+x)-carbon products. The condensation reaction enables a simple separation of the (5+x) carbon products from the residue because of their lower solubility in water. Some of the condensation products, such as levulinates, can undergo a second cyclic condensation (e.g. Dieckmann condensation) to produce cyclic ketones. In order to prevent significant self-condensation of the aldehydes or ketones, x can be chosen to be larger than 3. For providing suitable C9-C16 condensation products that are suitable for use in diesel and jet fuel after hydrotreating them, x can be in the range of 4 to 11.

In various embodiments, reagents for the condensation with the levulinic acid or ester thereof can be synthesized in a variety of ways from the separation products or by-products of the initial processing. In one embodiment (FIG. 1), ethanol from fermentation (700) of starches is converted to isobutyraldehyde (305) and used in the condensation reaction in the condensation unit (200).

In some embodiments, the sugars and starches can be used as a substrate for the production of hydroxymethylfurfural alkoxymethylfurfural, as well as alkyl levulinates (FIG. 2). In these reactions, in some embodiments, an aqueous or alcohol solution of the sugar or starch can be pumped through a bed of solid acid catalyst in the reactor (155). In an embodiment, the sugars are obtained by dilute acid hydrolysis from cellulose in unit (105) as illustrated in FIG. 2. In another embodiment, pectin is used to produce furfural; in such an embodiment the reactor (155) can include a pyrolysis unit that operates above about 200° C. and a separation unit to recover the furfural which is formed by decarboxylation.

The final integration can occur with the hydrotreatment of the condensation products; the hydrotreating unit (400) gives both linear and branched hydrocarbons of appropriate chain lengths for fuels, for example JP-8. In addition, cycloparaffins are available for example from Dieckmann and Diets-Alder reactions of the intermediates prepared from ethyl levulinate. Low molecular weight cyclic ethers from hydrotreating can be returned as solvent for the earlier separation.

In some embodiments (see, e.g., FIG. 1 and FIG. 2) a two stage hydrotreatment can occur. In some examples, a two stage hydrotreatment process can include a mild hydrotreatment in hydrotreatment unit (400) and a more severe hydrotreatment in hydrotreatment unit (600) with a separation step in separation unit (800) to recover MeTHF. In some embodiments, a two stage hydrotreatment process can improve yields of the desired product streams or give better control of the hydrotreatment. Additional products such as cyclic ethers and lighter carbon compounds of about C5 or less may also be removed in unit (800).

### 2. Initial Separation (Disassembly) (50, 55, 60)

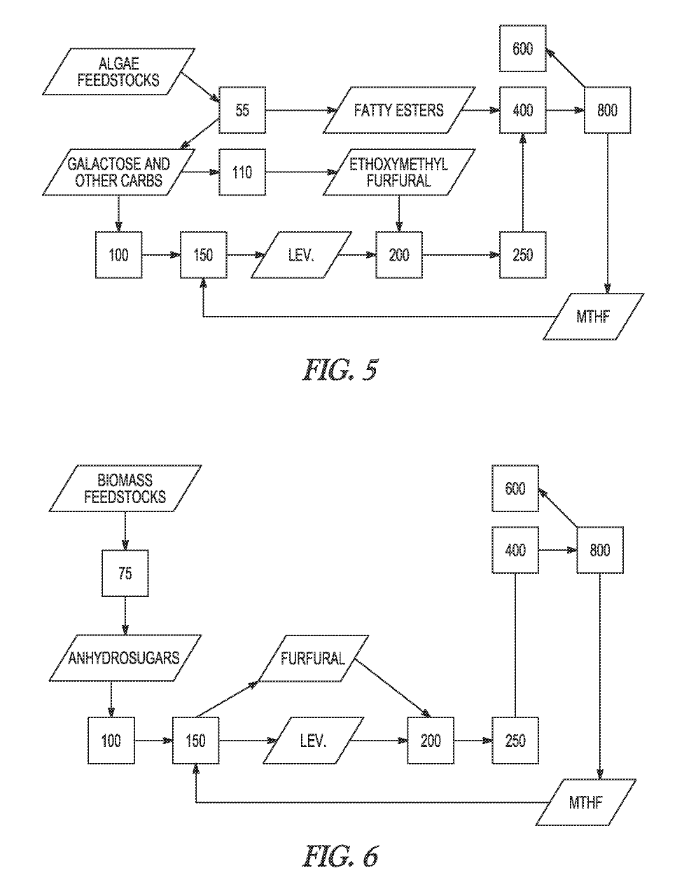

In an embodiment of this invention, where the feedstock is an oil seed such as corn, or the mechanical pretreatment unit (50) can be a wet mill which separates out the fibrous cellulosic material, from the starches and germ plasm, the germ plasm is treated by an oil extraction unit (55). The oil extraction unit (55) can be, for example, a press, or a hexane- or CO₂-based extraction unit (see FIGS. 1 and 5). Once the oil is extracted, it can be either directly hydrodeoxygenated in the hydrotreating unit (400) or first hydrolyzed to free fatty acids and glycerol in the hydrolysis unit (60), which is removed and further processed in unit (500), for example a purification unit. The starches and sugars can be fermented in a fermentation unit (700) to produce alcohols, e.g., ethanol. As illustrated in FIG. 1 the ethanol can be used to make isobutyraldehyde in unit (305). In an embodiment of this invention, the process consists of two steps. First, adding methanol to the ethanol isobutanol is formed via the Guebert reaction. Second isobutanol is dehydrogenated over a Cu catalyst to firm isobutyraldehyde.

When the feedstock is algae, as illustrated in FIG. 5, the extraction can be combined with transesterification to produce fatty acid esters, for example methyl or ethyl fatty acid esters.

When integrated with a Kraft process, as illustrated in FIG. 3, in one example the oil extraction unit (55) can yield tall oil fatty acids by separating the raw tall oil soap from the spent black liquor by decanting the soap layer formed on top of the liquor storage tanks and then performing extraction of the fatty acids. In an alternative embodiment, the tall oil soap is filtered. The extracted oil, fatty acids, or tall oil soap can then be hydrotreated in the hydrotreatment unit (400).

In another embodiment, the biomass feedstock includes a cellulosic or lignocellulosic material, such as wood, wood pulp, pulping sludge, particleboard, paper, grasses, agricultural by-products such as straw, stalks, cobs, beet pulp, seed hulls, bagasse, or algae, any of which could be a by-product or waste form of the material (see FIGS. 3-6). These are reduced to a small, preferably granular size for the catalytic processing through a mechanical pretreatment unit (50). This pretreatment can be any suitable pretreatment, for example, a simple mill or steam explosion gun. In another embodiment, the milled lignocellulose is further heated rapidly in a reactor (75, FIG. 6) to produce a condensable product including anhydrosugars, furfural, and lignin-based oils, which can be separated.

### Catalytic Depolymerization/Dehydration Unit (100)

Efficient processing of lignocellulosics to hydrocarbon fuels can include the removal of the large amount of oxygen without carbonizing or polymerizing the carbon structures and without expending unnecessarily large amounts of hydrogen. The present invention takes advantage of an acid-catalyzed mild thermal processing which can maintain chemical functionality useful for further synthetic reactions.

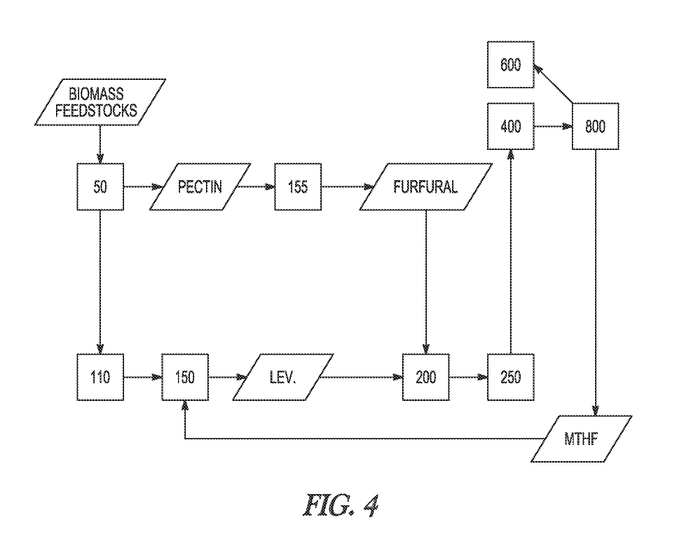

A catalytic depolymerization/dehydration unit can be a heated reactor (100), with temperatures of for example about 120° to about 200° C., shown in FIGS. 1-6. FIGS. 1-3 include a catalytic depolymerization/dehydration unit with a liquid or dissolved form of catalyst (for example, sulfuric acid). A heated reactor with a solid acid catalyst bed is utilized in FIGS. 4, 5, and 6. Feedstock for producing levulinic acid or esters thereof can be any suitable source of C6 sugar, for example cellulosic materials and starches. Examples of sources of C6 sugars that may or may not be pretreated include wood, wood pulp, pulping sludge, particleboard, paper, grasses, agricultural by-products such as straw, stalks, cobs, beet, beet pulp, seed hulls, bagasse, algae, corn starch, potato waste, sugar cane, and fruit wastes, any of which could be a by-product or waste form of the material or a combination thereof.

In some embodiments, integration with a power plant or a recovery boiler can furnish low-pressure (waste) steam to generate the desired temperatures for the different reactors.

The heated reactor can be, for example, a pressurized autoclave or a continuous reactor. In a continuous reactor a slurry of the biomass feedstock in acidic water or alcohol can be pumped or augured through the heated reactor under mild pressure and such that the residence time in the reactor is between 20 and 60 minutes.

In various embodiments, the catalytic depolymerization/dehydration unit can be run with an aqueous solvent or an alcohol solvent. In an aqueous medium, equal molar amounts of levulinic acid and formic acid can be produced, both of which are soluble/miscible in the aqueous acid. In examples including lignocellulosic material processing, furfural can also be formed (for example, from the 5-carbon units present in the hemicelluloses) and can be removed as overhead and collected during the processing. The furfural can be purified by distillation. Due to similar polarity, separation of the acid products from the aqueous acid solution or from each other can be difficult. However, for some acid-catalyzed processes, the process can continue through the next step without separation of the acids from the solvent because separation can be more easily effected on a more hydrophobic product from the subsequent reaction. In some examples, only the insoluble char and tars are separated, for example using a filter and solid- or liquid-phase extraction, respectively. In some examples, levulinic acid can be vacuum-distilled along with some of the water, or it can be extracted from the aqueous acid with an ether or ester solvent, such as a cyclic ether, e.g. methyltetrahydrofuran, which can be for example derived from the process in a later hydrogenation step. The insoluble char and tar can be further dewatered and can be thermally converted in a recovery boiler to provide process heat or fed to a power plant.

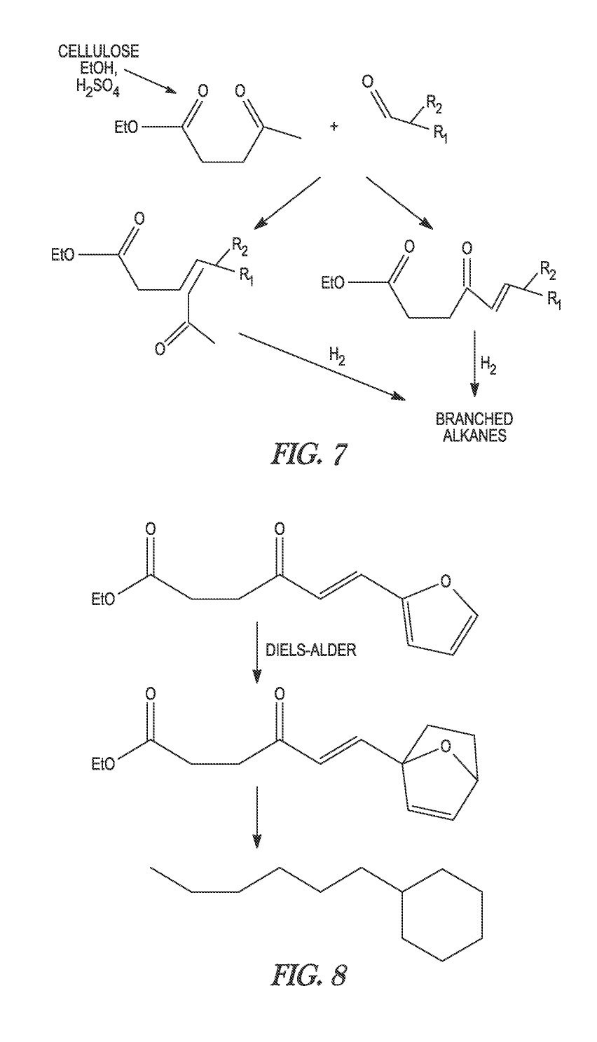

In another embodiment, the reaction medium for the depolymerization/dehydration can include an acidic alcohol solution, such as that obtained by adding sulfuric acid and methanol or ethanol. In some examples, ethanol can come from the fermentation unit (700). The products of the reaction include methyl levulinate and methyl formate (from methanol) or the corresponding ethyl esters (FIG. 7) (from ethanol). Longer-chain alcohols also can be used as the liquid medium, but in some examples they can give lower yields of the ester products. In some examples, the depolymerization/dehydration ethanol of particleboard and other waste materials to ethyl levulinate can proceed in good yield when conducted in ethanol with sulfuric acid catalyst at about 200° C. Compared to a similar preparation of levulinic acid using an aqueous acid medium, the levulinate can be more easily purified by (FIG. 10) extraction and/or distillation and can be easily separated from the concomitantly formed furfural (for example from the 5-carbon units present in the hemicellulose and ethyl formate). A preferred solvent for the extraction of levulinate esters and levulinic acid can be methyltetrahydrofuran, e.g. 2-methyltetrahydrofuran, in some examples produced in the hydrotreating unit (400) from ethyl levulinate or levulinic acid remaining in the condensation product mixture. Another preferred solvent is γ-valerolactone, which in some examples can also be produced in the hydrotreating unit (400) from the same source. In one embodiment, the extraction solvents can be provided by the integrated process; in other embodiments, the extraction solvents can be provided otherwise.

As shown in FIG. 10, in various embodiments the product mixture after pressure let down can be filtered and flash distillation in separation unit (150) can be used to remove materials including ethanol and ethyl formate. The residual from the distillation can include ethyl levulinate, furfural, lignin-derived phenolics, sulfuric acid, and water. The residual can be extracted with a polar solvent, such as with methyltetrahydrofuran, e.g. 2-methyltetrahydrofuran, to remove the ethyl levulinate and furfural. The remaining aqueous acid can be recycled to the first stage reactor. The organic phase can be utilized directly in the condensation reactor for conversion to fuel products, since it contains not only the desired reactants, but also a preferred solvent for the condensation reaction. To obtain pure products for chemical feedstocks, the organic phase can be extracted with aqueous base or a basic solid phase medium to remove the phenolic components resulting from the lignin decomposition. The solvent phase can be distilled to recover the solvent and vacuum distilled to give furfural and at higher temperatures, ethyl levulinate. If the furfural is not removed from the sulfuric acid by extraction, the acid can cause tar and carbon formation during distillation.

Another embodiment of the thermocatalytic unit (100) includes distillation of a levulinic acid product so as to give α-angelica lactone, also called 5-Methyl-2(3H)-furanone (see FIG. 2). The α-angelica lactone is highly reactive in subsequent condensation reactions, owing to the acylation reactivity of the enolic lactone group, and also provides a route to products substituted at the alpha position.

In another embodiment of the thermocatalytic unit (100), the depolymerization/dehydration is conducted at a lower temperature, wherein ethoxy (or methoxy) methylfurfural is formed in addition to the levulinic acid or ester thereof. In examples, this intermediate can be used directly in the condensation reactor or can be converted to chemical products and monomers, such as furan dicarboxylate. As illustrated in FIG. 5, a second thermocatalytic unit (110) can be introduced to conduct the depolymerization/dehydration at an even tower temperature in the presence of an acidic ion-exchange resin, so that predominantly ethoxy (or methoxy) methylfurfural can be formed and later utilized in the condensation unit (200).

### 4. Condensation Unit (200)

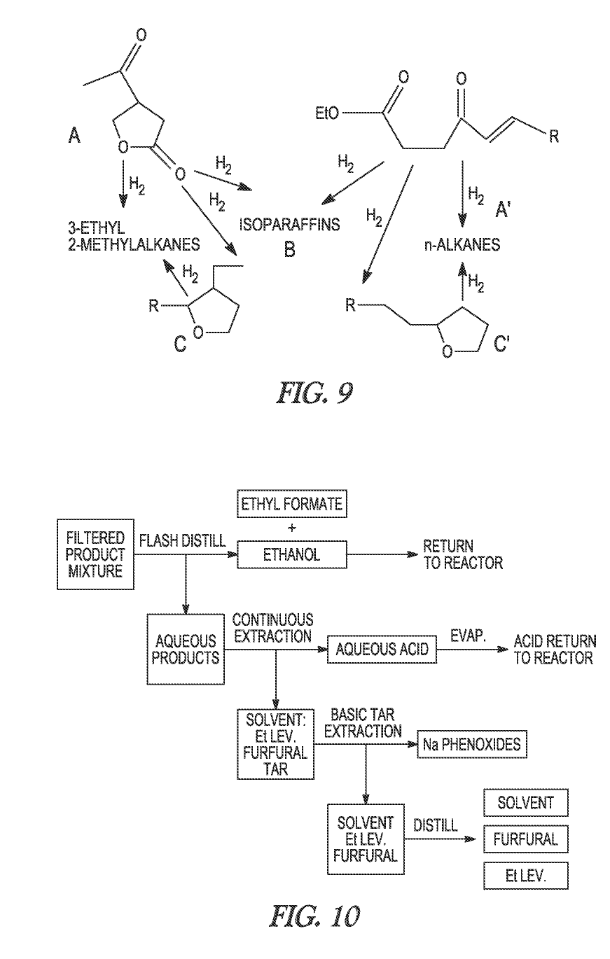

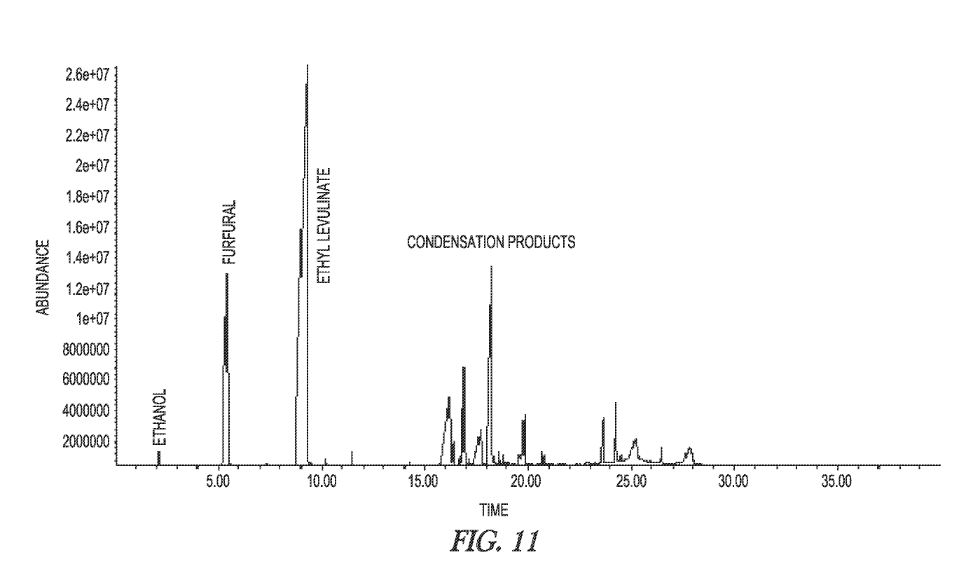

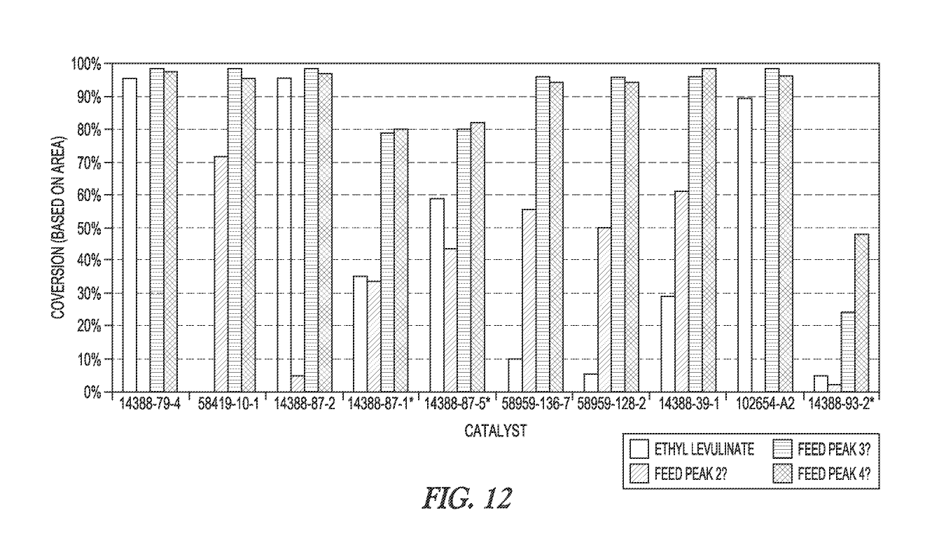

The condensation unit (200) in the integrated system is the reactor for conducting acid- or base-catalyzed condensation reactions of the levulinic acid or ester thereof to produce higher molecular weight species with the chain lengths desired for jet fuel, diesel, solvents and plasticizers. Thus the 5-carbon acyl group of the levulinic acid or ester thereof is combined with aldehydes, esters, or ketones (each with Cₓ) to form (5+x)-carbon products. The condensation reaction is illustrated in FIG. 7 with ethyl levulinate. In FIG. 7, a branched aldehyde. (wherein R1 and R2 can be the same, different, or linked to form a ring) condenses with ethyl levulinate to form a mixture of branched unsaturated ketoesters which can then be hydrogenated to give branched alkanes. Hydrogenation of the branched unsaturated ketoester can allow formation of cyclic ethers as shown in FIG. 9. In order to prevent the aldehydes, ketones, or esters from undergoing a predominantly self-condensation reaction, which is primarily an issue with certain aldehydes but can occur with ketones or esters also, compounds with reactive carbonyl groups can be chosen to have relatively unreactive alpha carbons, for example wherein the alpha carbon is branched or aromatic at this position, e.g. x can be greater than about 3 in some examples. For providing suitable C9-C16 condensation products that are suitable for use in diesel and jet fuel after hydrotreating them, x should be in the range of about 4 to about 11. In addition to x values between about 4 and 11, the aldehydes, esters, or ketones can be branched at or near the alpha carbon to help reduce the potential of self-condensation. Also, for producing jet fuel, in some examples branched or aromatic aldehydes, esters, or ketones can be preferred to help produce a highly isoparaffinic fuel blendstock or cycloparaffinic fuel blendstock, that when blended together meet such important jet fuel criteria as freeze point, flash point, energy density, and physical density.

In various embodiments, a product fuel or fuel component can include an appropriate distribution of carbon chain lengths to provide a with certain characteristics or fuel components that can form fuel mixtures with specific characteristics or, for example, to provide a convenient distillation curve for the fuel or fuel component. In certain embodiments, a product solvent can include an appropriate distribution of carbon chain lengths such that the solvent has the appropriate polarity characteristics, for example a polar, amphiphilic, or nonpolar character. In some examples, a product plasticizer can include an appropriate distribution of carbon chain lengths to provide a plasticizer that can allow formation of, for example, a polymer with highly elastic features. Therefore, the reagent aldehydes, esters, and ketones can be derived from a group of feedstocks and chemical reactions that can give the required carbon chain length distribution in the product or products. Examples of feedstocks for the reagent branched aldehydes include alcohols, such as isobutyl alcohol, which can be produced by Guerbet reactions of ethanol and subsequently dehydrogenated to aldehydes, and olefins, for example from a petroleum refinery, that can be converted to aldehydes by an oxo reaction. Examples of suitable aryl aldehydes include furfural, hydroxymethylfurfural, and substituted benzaldehydes that are produced from 5 and 6 carbon sugars or from lignin, respectively. Examples of suitable cyclic aliphatic aldehydes include those produced by Diels-Alder reactions of acrolein (from, for example, dehydration of glycerol) with butadiene (from, for example, petroleum cracking or from ethanol via the Lebedev reaction). Examples of suitable reactive ketones include those with an adjacent carbonyl (1,2 diketones, 1,2 ketoesters) that are produced by fermentation or pyrolytic reactions of levulinic or, lactic acid. Examples of suitable esters include vinyl esters, which can be highly reactive reagents; one example of a suitable ester is α-angelica lactone which can be, for example, produced by distillation of levulinic acid over a mineral acid.

Condensation reactions of levulinic acid or levulinic acid esters have precedence in the chemical literature, but these isolated reactions were not recognized for the potential of synthesis of fuel or fuel additives. Examples include the following: benzaldehyde and substituted benzaldehydes (Erdmann, H. Ber. Deutschen Chem. Gesellschaft 1891, 24, 3201-3204; Kato, H.; Ojima, H. Aichi Gokugel 1958, 7,25-2g; Sen, R. M.; Biresh, C. J. Indian Chem Soc. 1930, 7, 401-416; Borshe, W. Ber, Deutschen Chem. Gesellschaft 1915, 48, 842-849), furfural (Ludwig, K. Ber. Dentschen Chem. Gesellschaft 1891, 24, 1776-8; Sen, R. M.; Biresh, C. J. Indian Chem Soc. 1930, 7, 401-416; Erdmann, H. Ber. Deutschen Chem, Gesellschaft 1891, 24, 3201-3204), isobutyraldehyde (Meingast, F. Monatchefte fur Chem. 1905, 26, 265-277), and self-condensation (Zotchik, N. V.; Miroshnichertko, L. D. Evstigneeva, R. P.; Preobrazhenskii, N. A. Zhurnal Obshchei Khimii 1962, 32, 2823-8; Blessing WO 2006/056591), formaldehyde (Olsen, S. Acta Chem Scand. 1955, 9, 101) and phenol (Mauz, O. Justus Liebigs Ann. der Chemie 1974, 3, 345-351). A recent patent application teaches the dimerization of levulinic acid on a cation exchange resin to form C10 units (Blessing, WO2006/056591). The reaction proceeds in very low yields, 15% as reported. An older publication reports essentially the same process with a simple sodium base (Zotchik). In contrast, embodiments of the present invention utilize an integrated process where levulinate esters are condensed with aldehydes in high yields and the condensation products are converted to, for example, fuels, fuel components, solvents, or plasticizers.

Product formation and separation can be facilitated at the condensation stage because of the lower solubility of the longer-chain reaction products in water; this can be particularly beneficial when an aqueous medium is used in the thermocatalytic treatment step. Thus, in one example, when levulinic acid from a thermocatalytic aqueous reaction containing the acid catalyst is reacted with the aldehyde mixture (e.g. C4-C11 aldehydes) or similarly levulinic acid is with C4-C11 ketones, C4-C11 esters, or C4-C11 ketoacids, for example levulinic acid, the products from the condensation unit (200) can be more easily extracted from the water, for example using the solvent methyltetrahydrofuran. The acidic aqueous layer can contain formic acid in addition to the sulfuric acid. The formic acid can be vacuum-distilled along with some of the water in a separation unit (250), and the sulfuric acid catalyst can then be recycled back to the dissociation/depolymerization unit. Prior to recycling, water can be at least partially evaporated from the sulfuric acid. Thus the integration of aqueous thermocatalytic treatment and condensation steps can allow for convenient product separation as well as a means of recycling an acid catalyst. In some examples, no neutralization is needed. Aldol condensation products from the reaction of levulinic acid and an aldehyde, ketone, ketoacid, or ester conducted with an acid catalyst can be a mixture of the β-(or branched) and the δ-(or unbranched) forms, as shown in FIG. 7. In some examples, to achieve more of the δ-(or unbranched) form, a basic catalyst can be used; generally, this requires removing an acid catalyst (such as sulfuric acid) used in the thermocatalytic treatment unit. Thus an alternative route can be used when employing an alkaline catalyst for synthesis of predominantly unbranched isomers. Although in examples some of the aldehyde can undergo a self-aldol condensation, in some examples the products from this side reaction are not removed since they can be also converted to usable fuels or other products in the hydrotreatment step.

In an example that includes use of alcoholic medium in the thermocatalytic treatment stage, the esters formed from tile thermocatalytic treatment can be extracted and then, for example, separated by simple distillation having a lower boiling point, the formate ester and the alcohol can be removed first, followed by furfural. The higher boiling levulinate ester can be distilled or reacted without purification.

In some examples, the levulinate ester that is formed in a depolymerization/dehydration unit using alcoholic medium can be reacted with an aldehyde using a strong base catalyst to produce mainly longer-chain esters. In some embodiments, the catalyst for the condensation is a solid base catalyst so that a continuous reaction over the bed of the catalyst is performed, and no catalyst separation or neutralization is needed. In some embodiments, the solid catalyst is a hydrotalcite, such as for example a hydrotalcite impregnated with a basic material, such as potassium fluoride. When a soluble catalyst is employed, removal of the catalyst from the product solution can be significantly more involved than removal of a solid catalyst.

In examples, the condensate product mixture includes a mixture of isomeric forms. For example, isobutyraldehyde can be attacked by enolate carbanions formed at the delta or beta positions of a levulinic acid or levulinic acid ester. The proportion of isomers can depend on the catalyst used.

In another embodiment, a furfural by-product or coproduct can be condensed with the levulinic acid or ester thereof to form a furfuryl-substituted levulinic acid or ester thereof. In various embodiments, the furfuryl-substituted levulinic acid or ester thereof can then undergo a Diels-Alder reaction, the product of which can be subsequently hydrogenated and reduced (FIG. 8). Again, depending on the choice of catalyst, various mixtures of β-(or branched) and the δ-(or unbranched) isomers can be obtained. Hydroxymethylfurfural can react at the aldehyde moiety with levulinic acid or esters thereof to give a C11 intermediate. In one example, hydroxymethylfurfural is available from processing sugars with acid catalysts. Fructose has been the preferred sugar substrate for conversion to hydroxymethylfurfural; however, recent reports use CrCl₂ catalyst with glucose, for example which can be used in process unit (800). In another embodiment, furfural or another aldehyde with a diene can undergo a Diels-Alder reaction prior to condensation with levulinic acid or an ester thereof, which can then undergo hydrotreatment as described herein.

Several options are available for processing of the furfuryl-substituted levulinic acid or esters thereof. One option includes mild hydrogenation to give tetrahydrofurans. Another option is to open the furan ring to produce C10 or C11 units. In another option, a cycloaddition at the furan functionality, FIG. 8, can occur with a dienophile such as acrolein or acrylic acid (Diels-Alder reaction). The cycloaddition product contains the 7-oxa-bicyclo {2.2.1} heptene moiety with a bridging oxide group that can be subsequently removed in the hydrogenation step (400).

In some embodiments, α-angelica lactone prepared in certain embodiments of the thermocatalytic treatment can be condensed with the aldehyde mixture. The resulting products from this reactant can be substituted in the alpha position and can generate isoparaffins in the hydrogenation reactor (400).

In some examples, highly reactive ketones can condense with the levulinic acid or an ester thereof. Examples include biacetyl (2,3-butanedione) and 2,3-pentanedione. In some examples, both can be obtained from other reactions of levulinic acid. These highly reactive ketones can condense with levulinic acid or esters thereof giving C9 or C10 chains, respectively. Other branched and cyclic ketones are available, for example, from pyrolysis of lignin.

Another embodiment utilizes the condensation of levulinic acid or esters thereof with α-angelica lactone using a Lewis acid catalyst. The reaction can occur between an enolate of the levulinic acid or ester thereof and an activated carbonyl group to produce a diketone product. The condensation of α-angelica lactone with aldehydes can also occur. Condensation with the aldehyde can occur at the alpha position due to activation of the alpha position with basic catalysts.

Another embodiment utilizes the condensation of levulinic acid or an ester thereof with an unsaturated carbonyl compound, such as ethyl acrylate or acrolein, where an alpha carbon of the levulinic acid or ester thereof reacts with the beta carbon of the unsaturated carbonyl compound (e.g. Michael reaction). Some examples can include a catalyst to activate the unsaturated carbonyl compound or to promote enolization of the levulinic acid or ester thereof, for example a coordinating metal catalyst, for example a catalyst including zinc, nickel, and other transition metal ions, as well as titania, alumina, and zirconia.

Another embodiment produces cyclic ketones for example via a Dieckmann condensation of derivatives of levulinic acid or esters thereof. These cyclic ketones have the advantage that they are easily hydrogenated to cycloparaffins without formation of cyclic ethers.

In some embodiments, a condensation method combines an olefinic group with a carbonyl compound. In one example, reaction of manganese(III) acetate with the carbonyl compound can generate a free-radical, which subsequently combines with the olefin. With levulinic acid or esters thereof, two examples include: 1) reaction of ethyl levulinate radical with an added olefin or 2) reaction of an added ester with the double bond of α-angelica lactone which can be produced in a prior dehydration reaction from either levulinic acid or levulinate ester.

An acid catalyst for the condensation step can include any suitable acid catalyst. For example the catalyst can include an acid such as sulfuric acid or phosphoric acid. In some embodiments, the catalyst includes a solid catalyst to facilitate separation. In some examples, the solid catalyst can include any supported acid. For example, the catalyst can include any suitable sulfonated or, phosphonylated polymer, such as Nafion, PBI-sulfonate, or an inorganic strong solid acid, such as sulfated metal oxides (sulfated zirconia) or niobic acid. In some embodiments, the solid catalyst can include a transition metal or a heterogeneous catalyst including at least one of a sulfated catalyst and a sulfonated catalyst. In some embodiments, a sulfated catalyst can include sulfated titania, sulfated zirconia, sulfated alumina. In some embodiments, a sulfonated catalyst can include sulfonated activated carbon, sulfonated mesoporous carbon, sulfonated carbon composite, or sulfonated polymer.

In some examples, the catalyst can be reused with no ill-effect on the yield of subsequent runs. In some examples, the catalyst cannot be reused without a negative effect on subsequent runs. In some examples, hydrotalcite catalysts can be reused with minimal negative effect on subsequent runs. In some examples, hydrotalcite catalysts can be calcined to restore their catalytic abilities, allowing them to be reused with minimal effect on yield.

### 5. Reagent Aldehyde and Ester Production Unit

Various aldehydes are available from a variety of renewable or petrochemical resources. As discussed herein, the use of an aldehyde that undergoes minimal or no self-condensation can be advantageous. The class of minimally self-condensing aldehydes includes aldehydes with no hydrogen atoms on the alpha carbon, such as furfuraldehyde and benzaldehyde. Other examples include aldehydes with branching at the alpha carbon, such as isobutyraldehyde and cyclohexanecarboxaldehyde, the steric hindrance at the alpha position of which can inhibit self-condensation.

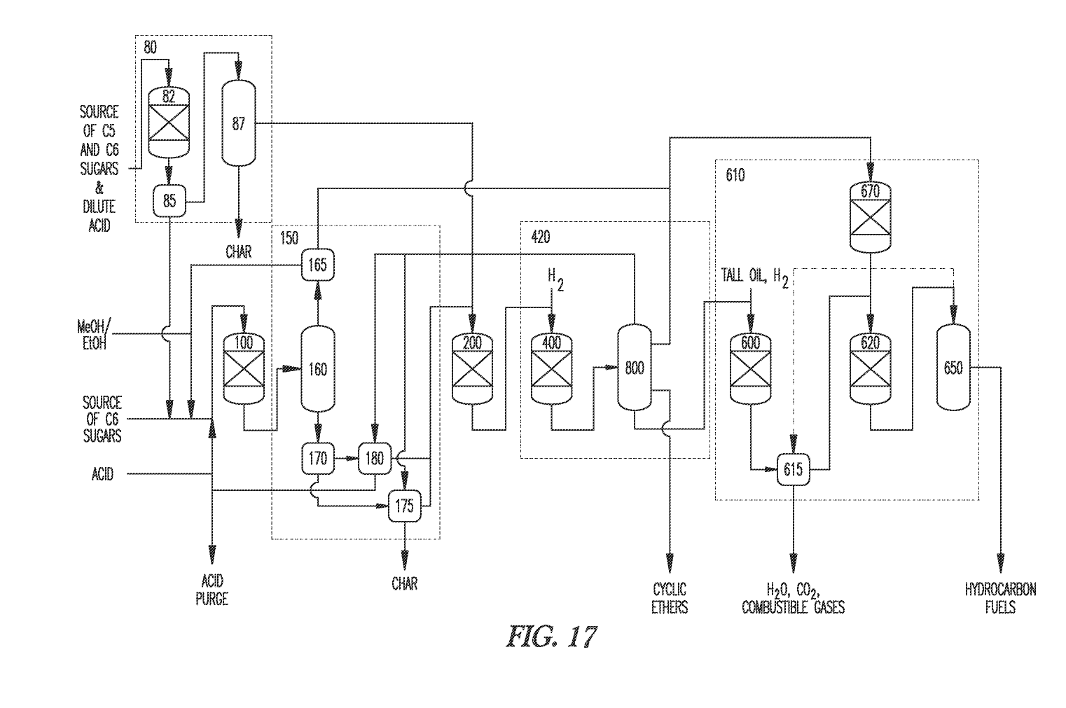

In the present invention, the reagent aldehydes can be supplied or produced by any suitable process. In some examples, reagent aldehydes can be formed by dehydration of alcohols over a catalyst that includes Cu or Pt. In some examples, precursor alcohols can be prepared via Guerbet synthesis or by homologation of tower alcohols with carbon monoxide. In some examples, aldehydes can be prepared directly from lower alcohols by Guerbet synthesis at higher temperatures (>400° C.). Isobutanol can be prepared from ethanol and methanol using a solid basic Guerbet catalyst, for example in process unit (305) as illustrated in FIG. 1. In another example, isobutanol can be produced from H₂ and CO under high pressure conditions. A variety of higher alcohols are present in fusel oil, a by-product from distillation of ethanol from yeast fermentation. In some examples, isobutyraldehyde can be prepared commercially by oxo reactions of propylene. In some examples, furfural can be produced from the thermal decomposition of 5-carbon sugars. As illustrated in FIG. 3, the separation unit (55) in a Kraft mill can include a separation of C5 sugars that are subsequently turned into furfural in the presence of a solid acid catalyst. A more detailed illustration of the process is given in FIG. 17, including the mild hydrolysis unit (82), cellulose separation unit (85), and furfural conversion unit (87). In some examples, alkoxymethylfurfural can be produced from the acid-catalyzed depolymerization cellulose and starch, for example at lower temperatures. Cyclohexenylcarboxaldehydes can be produced by the cycloaddition of acrolein (for example from glycerol or lactic acid) with butadiene, from example obtained from the condensation of ethanol (Lebedev process), or for example obtained via the reaction of acetaldehyde with an olefin (Prins reaction). In some examples, C6 and C9 aliphatic aldehydes can be formed from oxidation of fatty acids or triglycerides, such as tall oil fatty acids when integrated with the Kraft process. Benzaldehydes are available from a variety of renewable sources and for example by the oxidation of lignin. In some examples, benzaldehydes can be provided by the hydroformylation of BTEX (benzene, toluene, ethylbenzene, and xylenes). BTEX can be provided by, for example, biomass or coal gasification tars or from petroleum. In some embodiments, lignin can be recovered from solids separated in unit (150) and thermally processed to form BTEX. In one embodiment, benzaldehydes or substituted benzaldehydes can be allowed to condense with levulinic acid or esters thereof, and the condensation product thereof can be hydrotreated to give a product mixture that includes cycloparaffins.

Michael reactions can be conducted with ethyl acrylate, which can be for example obtained from dehydration of ethyl lactate. In some examples, lactic acid from fermentation of starches can be esterified in unit 200. Ethyl lactate can be converted catalytically to ethyl acrylate, which can undergo a Michael reaction in the condensation reactor 200.

In one embodiment, aromatic carboxaldehydes can be condensed with levulinic acid or esters thereof. The aromatic carboxaldehydes can be provided by, for example, hydroformylation of BTEX. The resulting condensation products can be hydrotreated to provide cycloparaffins, for example, for blendstocks. In some embodiments, the hydrotreatment does not completely hydrogenate all the aromatic rings, leaving a small percentage (for example, 1%, 5%, 10%, 20%, or about 30% by wt %) as alkylaromatics.

### 6. Catalytic Hydrogenation Units

The present invention can include a hydrotreatment step, which can include hydrogenation, reduction, or any suitable chemical process understood by one of skill in the art to be included in hydrotreatment.

A catalytic hydrogenate, can be performed on ketoacid- or ketoester-containing intermediates produced in the condensation unit (200). The oxygen functional groups can be reduced with unsaturation, which can result in formation of the mixtures of paraffins, isoparaffins, cycloparaffins, and alkylaromatics in a hydrogen atmosphere in the hydrogenation reactor (400). Under milder conditions, a tetrahydrofuran ring can form. Substituted tetrahydrofurans can be utilized as solvents or can be blended with hydrocarbon fuels or alcohol-based fuels.

Hydrotreatment of the C6-C8 condensation products, for example using an catalyst such as an isomerization catalyst, can give branched hydrocarbons, which can be suitable for gasoline or gasoline additives.

Hydrogenation of the C9 to C14 condensation products can give both linear and branched hydrocarbons of appropriate chain lengths for kerosene or kerosene additives, which can allow for example the production of jet fuel such as Jet A, Jet A1, JP-5, and JP-8. In addition, cycloparaffins can be available from Diels-Alder reactions of intermediates prepared from ethyl levulinate.

In some embodiments, it can be advantageous for fuel properties or processing for trialkylglycerides or tall oil fatty acids extracted in the oil extraction unit (55) to be directly processed by the hydrogenation reactor (400) together with the condensation products. Similarly, turpentine extracted from the Kraft process can undergo an aromatization reaction of its main terpene with reagents such as iodine or PCl₃, leading to cymene which then can then be hydrotreated to cycloparaffin.

In some embodiments, it can be advantageous to introduce a two stage hydrotreatment process which includes a mild hydrotreatment in hydrotreatment unit (400) between 200° C. and 300° C. more preferably at around 250° C., and a more severe hydrotreatment hydrotreatment unit (600) between 250° C.-400° C., more preferably around 350° C., with a separation step in separation unit (800) to recover a cyclic ether such as, for example, MeTHF. Additional products such as carbon compounds of about C5 or less can also be removed in unit (800).

### 7. Chemical Synthesis Units

Solvents, such as for example methyltetrahydrofuran or other cyclic ethers or other suitable solvents that can be derived from the levulinic acid of levulinic acid ester condensation reactions, can be used to conduct extractions in the method, for example to extract levulinic acid or esters thereof from the other reaction components. In some examples, methyltetrahydrofuran and other furan-derived products can also be utilized to extract fermentation products from their aqueous solutions. Thus butanol present in water, for example in tow concentrations, can be extracted from the aqueous fermentation broth. Recovery of butanol from the extraction solvent can occur via distillation if the boiling point of the extracting solution is sufficiently higher than that of the butanol. Solvents that can be derived from the present process include, for example, polar, amphiphilic, or non-polar solvents.

Several synthesis steps can be incorporated into the integrated parallel processing plant design that can utilize intermediate reagents produced from the noncellulosic feedstocks as well as the levulinic acid or esters thereof from the cellulosic feedstock. One of the embodiments is the use of a tong-chain unsaturated fatty ester, such as an oleate, in the condensation units (200) with levulinic acid or esters thereof to produce a long-chain ketoester. In some examples, the condensation reaction employed is the free radical condensation with the unsaturated portion of an unsaturated or polyunsaturated fatty ester. In some examples, this gives a product ester with a very low vapor pressure that has an appropriate mixture of flexible alkyl chains and polar groups which allows it to act as a plasticizer. In some examples, the product can dissolve in and plasticize a polymer material, such as for example vinyl chloride. In one example, the fatty esters can be produced in a transesterification unit from extracted vegetable oils or algal oils.

Another embodiment includes an acid-catalyzed reaction of levulinic acid or an ester thereof with a diol or polyol to produce a cyclic ketal (e.g. a 1,3-dioxolane or 1,3-dioxane). In one example, ethylene glycol, propylene glycol, or a glycerol monoether or glycidyl ether derived from the noncellulosic biomass can be used, to give a dioxolane, alkyldioxolane, or an alkoxymethyl-substituted dioxolane. Other polyol reagents can be derived from alkoxy sugars. In some examples, when an alkyl or alkoxy group in the dioxolane product is long, the vapor pressure is low, and good plasticizer properties are obtained. When an alkyl or alkoxyl group in the dioxolane product is short (for example, H, methyl, ethyl), in some examples the dioxolane product can serve as an intermediate for chemical synthesis, such as condensation reactions to give 2-substituted acrylates. In some embodiments, for the case of dioxolanes derived from diols, a dioxolane ester can be reacted with a glycerol to form a glyceride that can be valuable for polyester and polyurethane synthesis. In some examples, the glyceride can be allowed to react with a carbonyl compound, such as formaldehyde or acetone, to restore the ketone group of the levulinate glyceride. The reaction is driven by distillation of the small dioxolane, which can then be utilized as a diesel or gasoline additive, depending on the size and number of the alkyl groups attached.

In some embodiments, the reaction of levulinate or levulinic acid with the glycol or glyceryl derivative in the above examples can utilize the crude mixture obtained directly from the cellulose depolymerization/decomposition, as well as any sulfuric acid present in the mixture. The separation of the product from an aqueous phase (by, for example, simple decantation) can be facilitated by virtue of hydrophobicity conferred by a long alkyl group, if present.

Several options are available for integration with a Kraft pulp and paper mill. One such embodiment includes the use of wood and wood residues as a source of C5 and C6 sugars, and tall oil as input streams while providing some of the wood that is liberated of its hemicelluloses back to pulping process as well as char and purged acids to the recovery boiler for reducing the black liquor (FIG. 17). The embodiment can include the C5 sugar conversion unit (80), the depolymerization/dehydration unit (100), the condensation reactor (200), the first-stage hydrotreatment unit (420), and the fuel production and upgrading unit (610).

In the C5 sugar conversion unit (80), wood, which contains hemicelluloses and is a source of C5 sugars, can be mixed with dilute acid and provided to the mild hydrolysis unit (82) to liberate the hemicellulose. In contrast to cellulose, which is crystalline, strong, and resistant to hydrolysis, hemicellulose has a random, amorphous structure with little strength. It is easily hydrolyzed by dilute acid, such as sulfuric acid. A source of dilute acid can be the purge stream from the acid recycle coming of the separation unit (150) which also contains in addition to sulfuric acid acetic acid. Among pre-treatment methods for wood, dilute acid hydrolysis can be effective and inexpensive utilizing a continuous flow process, which can be carried out at a temperature higher than about 160° C. and used for about 5-10% (wt. of substrate/wt. of reaction mixture) loading. During hydrolysis the polysaccharides and monosaccharides of xylose and arabinose are formed and separated from the remaining solid materials containing the cellulose and lignin in separation unit (85), which can be a cyclone solid-liquid separator. The solids can then be either sent to the pulping process of the paper mill or utilized as a source of C6 sugar for the subsequent methanolysis/ethanolysis. The xylose/arabinose recovered from unit (85) can be converted into furfural and hydroxymethylfurfural by cyclodehydration using inorganic oxides such as zeolites and zeotypes, heteropolyacids, metal phosphates and sulfated metal oxides in the two-phase reactor or reactive distillation reactor (87); see, e.g., Mamman et al., Biofuels, Bioprod. Bioref. 2 (5): 438-454 (2008), and references cited therein. The remaining char can be extracted for use in the recovery boiler of the Kraft mill.

After suitable pretreatment a source of C6 sugars is provided and can be mixed with an alcohol, e.g. methanol or ethanol. The slurry can be subsequently mixed with a sulfuric acid catalyst and pumped to a catalytic depolymerization/dehydration unit (100) where the reactions can take place at temperatures of about 120° C. to about 200° C. The separation unit can include a flash distillation unit (160), a condenser (165), a char filter (170), a char washer (175), and an acid separator (180) which has the ability to purge spent acid catalyst and other acids such as formic acid. The product mix from the depolymerization/dehydration unit (100) can include methanol, dimethyl ether (DME), methyl levulinate, methyl formate, formic acid, sulfuric acid, char, water and smaller amounts of other organic compounds. The flash distillation unit (160) removes the DME, methyl formate, and methanol with the methanol being recovered for recycle in the condenser (165). The DME is further dehydrated over a zeolite catalyst, for example ZSM-5, in the dehydration unit (670) to give gasoline with typically 80% (by weight based on organics in the product stream) about C5 or longer hydrocarbon products. Other oxygen containing compounds separated from subsequent process steps can also be dehydrated in unit (670). The remaining products that are not flashed off in unit (160) can go through a set of additional separation steps. The char can be removed by the char filter (170). To recover more methyl levulinate the char can be washed with MeTHF in unit (175). The MeTHF from unit (175) can later be combined with the product stream that comes out of the acid separator (180). The acid separator can also utilize MeTHF to extract the methyl levulinate and smaller amounts of other organic compounds. The remaining acids and water can be recycled while small amounts can be purged and potentially concentrated with the purge stream being sent to the recovery boiler in the Kraft process. In the case of ethanol instead of methanol being used in the process, the product cycle is similar with the product mix from the depolymerization/dehydration unit (100) including, for example, ethanol, diethyl ether (DEE), ethylmethyl ether, ethyl levulinate, ethyl formate, formic acid, sulfuric acid, char, water and smaller amounts of other organic compounds.

The product stream leaving the separation unit (150) can be mixed with furfural and hydroxymethylfurfural from the C5 sugar conversion unit (80) and pumped into the condensation reactor (200) where it can form a variety of condensation products with reaction temperatures, for example, between about 20° C. and about 180° C., or between about 80° C. and about 150° C., including monoadduct and diadduct esters, monadduct and diadduct carboxylic acids, and the corresponding lactones as well as unreacted starting materials, MeTHF, and methanol or ethanol from hydrolysis of the esters.

The product stream can undergo a first-stage hydrotreatment in unit (420). The feed stream can be pumped into the mild hydrotreating unit (400) which is supplied with hydrogen. After hydrotreatment between about 200° C. and about 300° C., for example at around 250° C., the resulting product stream can include about 80-90% cyclic ethers, including MeTHF, and about 10-20% other organic compounds including predominantly C5 oxygen containing compounds, such as pentyl valerate and valerolactone, as well as traces of unreacted furfural and methyl or ethyl levulinate. Using preferably a vacuum flash distillation unit as separation unit (800), the MeTHF, which can be between about 20-30% of the total product mix, is recovered at around 90° C. and at least in part recycled for use in the separation unit (150). Excess MeTHF can be sent to the dehydration unit (670) or sold as a solvent. The remaining cyclic ethers can be recovered by flashing of the other organic compounds at around 120° C., which are sent to the dehydration unit (670). These cyclic ethers can be taken out of the cycle or can be further processed into hydrocarbon fuels.

Entering the fuel production and upgrading unit (610) as a feed stream, the long carbon chain cyclic ethers can be first hydrodeoxygenated hydrotreatment unit (600) between about 250° C. to about 400° C., for example around 350° C., in the presence of hydrogen. In some embodiments, at least one of free fatty acids, natural oils, tall oils, BTX (benzene, toluene, and xylenes), condensation products derived from BTX, and any combination thereof can be co-processed. In some embodiments, co-processing can improve the quality of the resulting product, such as for example by improving the final fuel characteristics. Catalysts for the second hydrotreating step leading to complete hydrodeoxygenation of the product stream are, for example, standard hydrotreating catalysts available from petroleum refining which can be used either sulfided or non-sulfided. In addition to hydrocarbon products, the products can include CO₂ and water, which can be removed in separation unit (615) along with some light hydrocarbon cracking products. In the case of using a sulfided hydrotreating catalyst, there can also be trace amounts of sulfur species. In order to maintain the sulfided catalyst, small amounts of sulfur containing compounds can be present before the hydrotreatment and can be removed for further processing and upgrading. Standard metal oxide adsorbents, such as a quadrilobe extrudate nickel adsorbent (e.g. BASF D-1275), remove various sulfur species (e.g. mercaptans, sulfides) from the product stream in a fixed bed reactor. The hydrocarbons can be further upgraded by running them into the isomerization unit (620). In addition, the C5+ hydrocarbon products exiting the dehydration unit (670) can be co-processed in the isomerization unit (620). In the final step the fuels can be separated into gasoline, jet fuel, and diesel in fractional distillation column (650).

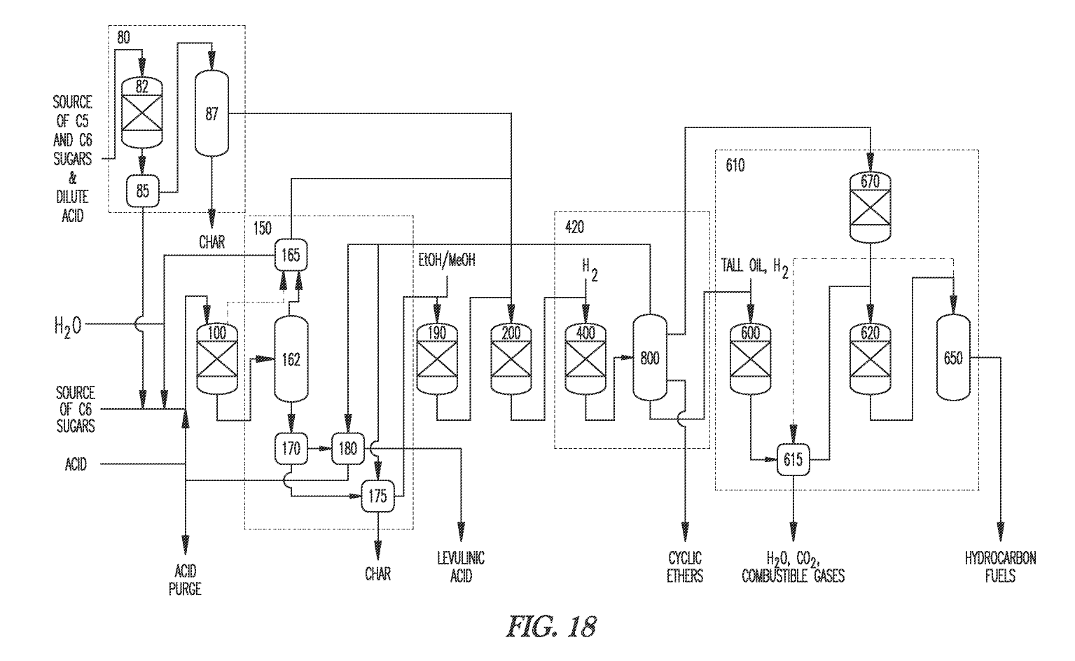

Instead of implementing the methanolysis/ethanolysis for depolymerizing cellulosic materials, the catalytic depolymerization/dehydration unit (100) can be run with an aqueous solvent mixed with a sulfuric acid catalyst. In an aqueous medium, levulinic acid and formic acid can be produced, in some embodiments the amounts produced are approximately equimolar, both of which are soluble/miscible in the aqueous acid. As illustrated in FIG. 18, the separation unit (150) then can include an evaporator (162), a condenser (165), a char filter (170), a char washer (175), and an acid separator (180) which has the ability to purge spent acid catalyst and other acids such as formic acid. The product mix from the depolymerization/dehydration unit (100) can include H₂O, levulinic acid, formic acid, sulfuric acid, char, and small amounts of other organic compounds. In examples including lignocellulosic material processing, furfural can also be formed (for example, from the 5-carbon units present in the hemicelluloses) and can be removed as overhead and collected during the processing and condensed in the condenser (165) or a separate condenser. The furfural can be either further purified and sold or used in a subsequent condensation reaction.

The evaporator (162) can concentrate the product stream by removing water with small amounts of furfural and formic acid to at least about a 30% or at least about 40% levulinic acid solution. At these concentration levels of levulinic acid the unexpected effect occurs that the levulinic acid can be separated from the sulfuric acid catalyst with MeTHF. It is generally known in the art that at low concentrations the separation is not effective.

Prior to separating the acid catalyst from the levulinic acid, the char can first be removed using the char filter (170). In another embodiment, the char filter (170) comes before the evaporator (162). To recover more levulinic acid the char can be washed with MeTHF in unit (175). The MeTHF from unit (175) can later be combined with the product stream that comes out of the acid separator (180). The acid separator can utilize MeTHF to extract the levulinic acid and other organic compounds. The remaining acids and water can be recycled. Recycling streams can be directly added to the feed stream to the evaporator (162) and/or to the feed stream to the depolymerization/dehydration unit (100) while small amounts can be purged for potential use as a dilute acid in pretreatment processes.

The levulinic acid product stream leaving the separation unit (150) can be further reacted in the esterification unit (190) with an alcohol, for example ethanol, methanol or a combination thereof, to produce esters such as ethyl levulinate or methyl levulinate. The levulinic esters can then be mixed with furfural and hydroxymethylfurfural from the C5 sugar conversion unit (80) or from the condenser (165) and pumped into the condensation reactor (200) where a variety of condensation products can be formed using reaction temperatures between about 20° C. and about 180° C., more preferably between about 80° C. and about 150° C., including monoadduct and diadduct esters, monadduct and diadduct carboxylic acids, and the corresponding lactones as well as unreacted starting materials, MeTHF, and methanol or ethanol from the esterification. Further processing to cyclic ethers and fuels can occur in the first-stage hydrotreatment unit (420), and the fuel production and upgrading unit (610).

Various modifications are possible to the herein described levulinate biorefinery process, and are encompassed as embodiments of the present invention. For example, the furfural can be replaced by aldehydes from carbonylation of olefins or BTEX. This can provide integration with a petroleum refinery or coal gasification plant. In such a case the char, for example, can be used for producing heat or sent to the gasifier, respectively. Instead of wood, agricultural waste products, such corn cobs, could be used as a source for both C5 and C6 sugars. In another embodiment, a sulfite for integration can be used instead of a Kraft mill. The starting material such as chipped wood can be digested in a sulfite digester. Pulp washers, using coutercurrent flow, can removespent cooking chemicals and degraded lignin and hemicellulose. The hemicelluloses can be separated and turned into furfural from the lignosulfonates in a counter-current hot-air column. The sulfite pulp can then be hydrolyzed in the catalytic depolymerization/dehydration unit (100) with the sulfuric acid catalyst as illustrated in FIG. 18. The yield of pulp can be higher than for Kraft pulping and sulfite pulp can be easier to hydrolyse reducing the amount of char.

### EXAMPLES

The present invention can be better understood by reference to the following examples which are offered by way of illustration. The present invention is not limited to the examples given herein, nor to any theories of operation given herein.

### Conversion of Cellulose and Carbohydrate Precursors to Levulinates

### Example 1.1A

### Reactions of Municipal Solid Waste (MSW) Cellulose—Ethanol

A dry cellulosic fiber was obtained from an MSW predigester process (provided by Tempico). The process was claimed to be effective in removing hemicellulose and lignin and removing some of the crystallinity of the cellulose. Reactions of the Tempico fiber (20 g) were conducted with ethanol and sulfuric acid in a 300-mL Parr reactor at about 200° C. A small amount of product gases were released after cooling and prior to opening the reactor. Analysis of the product by gas chromatography (GC) indicated a large production of ethyl levulinate (40%) with a small amount of furfural (as expected for a low hemicellulose content). Removal of the char (6.7 g) by filtration and ethanol solvent by distillation gave a residual composed of ethyl levulinate and water and sulfuric acid. The ethyl levulinate (9 g) was separated by adding 2-methylTHF which dissolved the ethyl levulinate, leaving aqueous sulfuric acid (3 mL). It is likely that the char contained a small amount of the sulfate, but it was not analyzed. The recovery of ethyl levulinate on a weight basis was 45%.

### Example 1.1B

### Reactions of Municipal Solid Waste (MSW) Cellulose—Aqueous

A reaction similar to Example 1.1A was conducted in aqueous sulfuric acid in the Parr reactor at 200° C. Again a small residual gas was observed. The char yield was 7.0 g. The weight % yield of levulinic acid as determined by HPLC analysis was 15%.

### Example 1.2

### Reaction of Shredded Paper—Aqueous

A reaction similar to Examples 1.1 with paper from a shredder was conducted in aqueous sulfuric acid in the Parr reactor at about 200° C. Again, a small residual gas was observed. The char yield was 6.7 g. The weight % yield of levulinic acid as determined by HPLC analysis was 28%.

### Example 1.3

### Reaction of Pineapple Waste—Aqueous

A sample of waste (rotten) pineapple was obtained and separated into a liquid and a wet, mushy material. The mushy portion (85 g) was heated in aqueous sulfuric acid (79 L) in the Parr reactor at about 200° C. The char product was removed (2.3 g). HPLC analysis of the liquid product indicated a yield of 13 wt % on a wet basis. It is likely that the mushy material included a mixture of sucrose and pectin. It is unlikely that the pectin converted to levulinic acid, and therefore most of the levulinic acid was derived from the sucrose present, rather than a polysaccharide, since there is not much cellulose or starch.

### Example 1.3A

### Reaction of Ethyl Cellulose—Aqueous

Reactions of ethyl cellulose (16 g) were conducted with water and sulfuric acid. The ethyl cellulose formed a slurry that was run in a 300-mL Parr reactor at 200° C. The char product was separated (2.8 g). HPLC determination of the levulinic acid gave 7% yield. The GC analysis indicated that ethyl levulinate was a major product (13%), accompanied by several unknown components in smaller amounts.

### Example 1.3B

### Reaction of Ethyl Cellulose—Ethanol

Ethyl cellulose was also reacted in an ethanol slurry in the Parr reactor at 185° C. The char product yield was similar (2.9 g). The GC analysis indicated 20% yield of ethyl levulinate.

### Discussion of Examples 1.1-1.3

The choice of feedstock for the acid-catalyzed depolymerization-dehydration of biomass wastes was shown to affect the yields of levulinic acid or esters thereof from the reaction. The reaction of shredded paper in aqueous acid gave a levulinic acid yield of 28% (weight basis), consistent with yields obtained previously in the same reactor with a waste wood composite.

The reaction with the cellulosic Tempico fiber derived from municipal solid waste (MSW) in aqueous acid was considerably lower. However, the yield of ethyl levulinate from the reaction in acidic ethanol was considerably higher, indicating an advantage to this type of processing. Isolation of ester product was relatively easy via distillation. The levulinic acid products or levulinic ester products were not actually isolated, but amounts in the aqueous sulfuric acid were determined by high-performance liquid chromatography (HPLC). The breakdown of the Tempico fiber occurs reasonably well in the ethanol solvent, and there is little lignin and hemicellulose to interfere with using this feedstock.

The yield of levulinic acid or esters thereof from the pineapple waste was lower. The reason is that the pineapple is composed mostly of pectin, which does not readily hydrolyze and decarboxylate 200° C. Most of the product is likely derived from the sucrose still present in the waste pineapple. There may be better uses for both the sucrose and the pectin. Sucrose can be converted to hydroxymethylfurfural. Pectin can be used in various ways, or pyrolyzed to furfural at higher temperatures than those employed in this step.

The depolymerization-decomposition of ethyl cellulose gave mostly ethyl levulinate as the product in either water or ethanol. In this reaction, the ethoxy groups in the ethyl cellulose are lost as ethanol during the reaction, just as water is lost from the glucose units of cellulose. It was hoped that the greater solubility of the cellulose derivative would result in much higher yields; however, this was not observed.

### Condensation of Levulinic Acid or Esters Thereof

### 2.1. Reactions of Furfural with Ethyl Levulinate.

The condensation of furfural was investigated with ethyl levulinate to form the furfuryl-substituted levulinates. Since furfural lacks alpha-hydrogens, it does not undergo a self-aldol condensation. It is also a by-product of the thermocatalytic reaction or produced independently from C5 sugars. Batch reactions were conducted in a 15-mL glass pressure tube (magnetic stirring) with a variety of catalysts at various temperatures. The pressure due to solvent vapor pressure was not determined. The results of these tests are indicated in Table 1 for basic catalysts and Table 2 for acid catalysts. Reactions were also performed in a 300-mL Parr autoclave with larger amounts of reagents. Results from these larger-scale batch reactions are reported in Table 3.

In Table 1 and 2, approximately 10 wt % of the catalyst was used, based on the total weight of the levulinate, exclusive of solvent, except that in Tables 1 and 2, about 0.5 grams of Amberlite-15H+ was used, and about 1 drop of H₂SO₄ (concentrated) was used. In table 3, about 8-10 grams of hydrotalcite catalyst was used, about 10 grams of Amberlite-15H+ was used, and about 1 mL of H₂SO₄ (concentrated) was used. In Tables 1-3, Sodium hydride was used as a 50% dispersion on mineral oil. Sodium ethoxide was a solution in ethanol. NaOH was used as pellets. MgO was used as a fluffy white powder. The hydrotalcite catalysts were obtained from a commercial vendor. HT-as rec indicated that the hydrotalcite was used as received. HT-cal550 or HT-ca550 indicates that the hydrotalcite was calcined at about 550° C. prior to use. HT-ca1450 or HT-ca450 indicates that the hydrotalcite was calcined at about 450° C. prior to use. phthHT-cal550 indicates hydrotalcite prepared using phthalic acid and calcined at 550 to remove the phthalate salt.

For the catalysts employed, β-(or branched) and δ-(or unbranched) isomers were obtained, as well as products with two furfuryl groups. For ethyl levulinate with a basic catalyst, initial β- or δ-condensation products containing an alcohol or alkoxy group can dehydrate to the unsaturated ester or can cyclize and displace the ethoxy group from the ester to produce a lactone (as in the Stobbe condensation). Ethanol is detected as a major component in the product mixture. The lactone can also open in the basic conditions to produce a carboxylic acid. The acids are largely undetected in the GC analysis owing to decomposition in the GC Condensation of two furfural units with one ethyl levulinate produces the β-, δ-difuryl ester.

TABLE 1
Reactions of Levulinic Acid or Esters Thereof with Basic Catalysts (sm.
pressure tube reactor).

| | | | Temp., | | | Conversion of |
|---|---|---|---|---|---|---|
| Reactant | Coreactant | Catalyst | ° C. | Time, h | Solvent | Eth. Lev. (%) |
| Ethyl Levulinate | Furfural | NaH | 25 | 12 | MeTHF | 67 |
| Ethyl Levulinate | Furfural | NaH | 60 | 4 | MeTHF | 88 |
| Ethyl Levulinate | Furfural | NaOH | 25 | 24 | Ethanol | 100 |
| Ethyl Levulinate | Furfural | MgO | 60 | 3 | None | 0 |
| Ethyl Levulinate | Furfural | MgO | 85 | 3 | None | 0 |
| Ethyl Levulinate | Furfural | HT-as rec | 60 | 3 | None | 0 |
| Ethyl Levulinate | Furfural | HT-as rec | 85 | 3 | None | 5 |
| Ethyl Levulinate | Furfural | HT-cal550 | 70 | 3 | None | 2 |
| Ethyl Levulinate | Furfural | HT-cal550 | 125 | 3 | None | 10 |
| Ethyl Levulinate | Furfural | phthHT- | 125 | 3 | None | 10 |
| | | cal550 | | | | |
| Ethyl Levulinate | Furfural | HT-cal550 | 150 | 12 | Diglyme | 33 |
| Ethyl Levulinate | Furfural | phthHT- | 150 | 12 | Diglyme | 60 |
| | | cal550 | | | | |
| Ethyl Levulinate | | NaOH | 60 | 2 | Ethanol | 0 |
| Ethyl Levulinate | Butyraldehyde | NaOH | 25 | 2 | Ethanol | 0 |
| Ethyl Levulinate | Butyraldehyde | NaOH | 60 | 2 | Ethanol | 0 |
| Ethyl Levulinate | Isobutyrald. | NaOH | 25 | 2 | Ethanol | 60 |
| Ethyl Levulinate | Valerolactone | NaOEt | 25 | 12 | Ethanol | 2 |
| Ethyl Levulinate | Me methacrylate | NaOEt | 60 | 3 | Ethanol | 2 |
| Ethyl Levulinate | Nitromethane | NaOEt | 60 | 3 | Ethanol | 0 |
| Ethyl Levulinate | Nitromethane | NaOEt | 150 | 3 | Ethanol | 0 |
| Ethyl Levulinate | Acrolein | NaH | 60 | 3 | Diglyme | 0 |
| Ethyl Levulinate | Acrolein | NaOH | 60 | 3 | Ethanol | 0 |
| Levulinic Acid | Furfural | NaOH | 60 | 1 | Ethanol | 0 |
| Levulinic Acid | Butyraldehyde | NaOH | 60 | 1 | Ethanol | 0 |
| Levulinic Acid | Isobutyrald | NaOH | 60 | 24 | Ethanol | 44 |

MeTHF = methyltetrahydrofuran.

TABLE 2
Reactions of Levulinic Acid or Esters Thereof with Acid Catalysts (small
pressure tube reactor).

| | | | Temp., | Time, | | |
|---|---|---|---|---|---|---|
| Reactant | Coreactant | Catalyst | ° C. | hr | Solvent | Conversion (%) |
| Ethyl Levulinate | Furfural | Amberlite-15H+ | 70 | 3 | None | 0 |
| Ethyl Levulinate | Furfural | Amberlite-15H+ | 100 | 3 | None | 100¹ |
| Ethyl Levulinate | Furfural | Amberlite-15H+ | 60 | 3 | None | 0 |
| Ethyl Levulinate | Furfural | Amberlite-15H+ | 60 | 2 | MeTHF | 0 |
| Ethyl Levulinate | Furfural | Amberlite-15H+ | 95 | 2 | MeTHF | 24 |
| Ethyl Levulinate | Furfural | PTSA² | 60 | 2 | MeTHF | 0 |
| Ethyl Levulinate | Furfural | PTSA | 95 | 2 | MeTHF | 30 |
| Ethyl Levulinate | Furfural | PTSA | 60 | 2 | MeTHF | 0 |
| Ethyl Levulinate | Furfural | PTSA | 60 | 2 | MeTHF | 0 |
| Ethyl Levulinate | Furfural | H₂SO₄ | 25 | 1 | None | 100¹ |
| Ethyl Levulinate | Furfural | H₂SO₄ | 25 | 12 | Ethanol | 0 |
| Ethyl Levulinate | Furfural | H₂SO₄ | 60 | 12 | Ethanol | 0 |
| Ethyl Levulinate | Acetald. | H₂SO₄ | 25 | 1 | Ethanol | 0 |
| Ethyl Levulinate | Butyrald. | H₂SO₄ | 25 | 2 | None | 0 |
| Ethyl Levulinate | Butyrald. | H₂SO₄ | 60 | 1 | None | 0 |
| Ethyl Levulinate | Isobutyrald. | H₂SO₄ | 25 | 1 | None | 63 |
| Levulinic Acid | Furfural | Amberlite-15H+ | 60 | 2 | None | 75 |
| Levulinic Acid | Furfural | Amberlite-15H+ | 100 | 4 | None | 81 |
| Levulinic Acid | Furfural | Amberlite-15H+ | 50 | 1 | MeTHF | 30 |
| Levulinic Acid | Furfural | PTSA | 60 | 4 | Diglyme | 68 |
| Levulinic Acid | Furfural | PTSA | 150 | 5 | Diglyme | 72 |
| Levulinic Acid | Acetald | H₂SO₄ | 25 | 24 | Ethanol | 0 |
| Levulinic Acid | Isobutyrald. | H₂SO₄ | 60 | 24 | Ethanol | 73 |

¹Carbonized, no products.
²p-Toluene sulfonic acid.

TABLE 3
Reactions of Levulinic Acid or Esters Thereof (300-mL Parr reactor).

| | | | Temp., | Time, | | Conversion |
|---|---|---|---|---|---|---|
| Reactant | Coreactant | Catalyst | ° C. | hr | Solvent | (%) |
| Ethyl Levulinate | Furfural | HT-cal.550 | 150 | 12 | None | 100¹ |
| Ethyl Levulinate | Furfural | HT-cal 550 | 150 | 12 | Diglyme | 74 |
| Ethyl Levulinate | Furfural | HT-cal550 | 125 | 12 | MeTHF | 30 |
| None | Furfural | HT-cal550 | 150 | 12 | Diglyme | 4 |
| Ethyl Levulinate | Furfural | HT-cal450 | 150 | 4 | MeTHF | 73 |
| Ethyl Levulinate | Furfural | Amberlite15 H+ | 120 | 4 | MeTHF | 100¹ |
| Ethyl Levulinate | Furfural | HT-ca450 | 125 | 14 | MeTHF | 72 |
| Ethyl Levulinate | Furfural | HT-ca450² | 135 | 4 | MeTHF | 70 |
| Ethyl Levulinate³ | Furfural³ | HT-ca450 | 135 | 14 | MeTHF | 70 |
| Ethyl Levulinate | | H₂SO₄ | 60 | 4 | None | 5 |
| Ethyl Levulinate | Isobutyrald. | HT-cal550 | 150 | 12 | Diglyme | 74 |
| Ethyl Levulinate | Isobutyrald. | NaOH | 60 | 4 | None | 29 |
| Ethyl Levulinate | Isobutyrald. | H₂SO₄ | 25 | 4 | None | 61 |
| Ethyl Levulinate | Isobutyrald. | H₂SO₄ | 60 | 4 | None | 26 |
| | Isobutyrald. | H₂SO₄ | 60 | 2 | None | NA |
| Ethyl Levulinate | Valerolactone | HT-cal550 | 150 | 12 | MeTHF | 0 |
| Ethyl Levulinate | Me methacrylate | HT-cal550 | 150 | 4 | MeTHF | 0 |
| Ethyl Levulinate | Acrolein | HT-cal450 | 150 | 6 | MeTHF | 2 |
| Levulinic Acid | Isobutyrald. | NaOH | 60 | 4 | None | 90 |
| Levulinic Acid | Isobutyrald. | H₂SO₄ | 60 | 4 | None | 30 |

¹Heavy tar product.
²Used.
³Double batch.

### Example 2.1.1

### Reactions of Ethyl Levulinate and Furfural with Liquid Base Catalysts

Small-scale reactions of ethyl levulinate with furfural in molar excess and liquid bases (e.g. dissolved bases) (NaH and NaOH) in solvents gave high conversions of the levulinate to condensation products (Table 1). The products are mixtures including the monoadducts and diadducts, and much of the product was hydrolyzed as in the Stobbe condensation. Some furfural remained in the products, but the ethyl levulinate was converted in the NaOH-catalyzed reaction.

A larger-scale reaction with NaOH with no solvent also gave a high conversion to products (Table 3). These results are consistent with those reported in the literature for reactions of ethyl levulinate with aromatic aldehydes.

### Example 2.1.2

### Reactions of Ethyl Levulinate and Furfural with Liquid Acid Catalysts

Small-scale reactions of ethyl levulinate and furfural gave generally poor conversions in liquid acid systems (H₂SO₄ and PTSA) (Table 2), although the neat reaction in H₂SO₄ formed a tarry polymer owing to overheating. The self-condensation of ethyl levulinate H₂SO₄ (no fufural) gave a poor yield of the condensation products.

### Example 2.1.3

### Reactions of Ethyl Levulinate and Furfural with Solid Base Catalysts

Small-scale reactions of ethyl levulinate with furfural using a solid MgO catalyst gave no conversion of the ethyl levulinate at low temperatures (Table 1). A commercial hydrotalcite catalyst gave poor conversions at tow temperature. A pillared hydrotalcite prepared using phthalate also gave poor conversions. Calcining the hydrotalcite did not improve the conversion. However, increasing the temperature to 150° C. improved the conversion by a major amount.

Several reactions of ethyl levulinate were then conducted in larger amounts in the Parr reactor with the commercial hydrotalcite catalyst calcined at 450° C. (Table 3). With no solvent, the reaction overheated and a solid tarry product resulted. Reactions conducted at 135° to 150° C. In either MeTHF or diglyme resulted in 70% to 74% conversions of the ethyl levulinate. The catalyst was reused in one run with no ill effect.

### Example 2.1.4

### Condensation of Ethyl Levulinate and Furfural in a Flow-Through Reactor

A granular catalyst was prepared from hydrotalcite (Mg:Al=2:1) by calcining the hydrotalcite at 450° C. for 3 hr. The calcined hydrotalcite was mixed with 20 wt % clay-molasses binder and dried in an oven at 110° C. A bed of the granules (size 1-2 mm) was prepared in a tube reactor from 50 g of hydrotalcite, and the tube was heated in at/be oven at 160° C. A solution of 1 mole ethyl levulinate plus 1 mole of furfural in 100 mL of methyltetrahydrofuran (MeTHF) was pumped through the reactor. The conditions included a liquid feed flow rate of about 0.5 mL/min, a temperature or about 160° C., a pressure or about 500 psi, and 19 g hydrotalcite catalyst.

The product was collected and analyzed by gas chromatography-mass spectroscopy (GC-MS). FIG. 8 shows a GC-MS chromatogram of product collected. The product included the monoadduct and diadduct esters, monadduct and diadduct carboxylic acids, and the corresponding lactones as well as unreacted starting materials and ethanol from hydrolysis of the esters.

### Example 2.1.5

### Reactions of Ethyl Levulinate with Furfural with Solid Acid Catalysts

Small-scale reactions of ethyl levulinate with furfural and Amberlite® 15 (H+ form) at 60°-70° C. (with or without solvent) gave no conversion (Table 2). However, at 100° C. and no solvent, the reaction gave a viscous, tarry product. In MeTHF, a small conversion was observed at 95° C.

### Example 2.2

### Reactions of Ethyl Levulinate with Other Aldehydes or Coreactants

Several other aldehydes were investigated as coreactants with ethyl levulinate. The most successful reactions were with isobutyraldehyde, since self-condensation was minized with this reagent.

### Example 2.2.1

### Reactions with Base Catalysts

Isobutyraldehyde was condensed with ethyl levulinate in the small-scale reactor. Using NaOH in ethanol solvent a 60% conversion of the levulinate ester was obtained (Table 1). As with the furfural, a variety of products were obtained. In contrast, butyraldehyde resulted in no conversion of levulinate, only self-condensation. Very little reaction was obtained for valerolactone, methyl methacrylate, nitromethane, and acrolein (Table 1).

Several base-catalyzed reactions were conducted with larger amounts of isobutyraldehyde and ethyl levulinate with base catalysts in the Parr reactor (Table 3). Reactions of isobutyraldehyde and ethyl levulinate with NaOH at 60° C. gave 29% conversion of ethyl levulinate. With the commercial hydrotalcite calcined at 450° C., the reaction of isobutyraldehyde in diglyme at 150° C. gave 74% conversion.

Valerolactone, methyl methacrylate, nitromethane, and acrolein were reacted with ethyl levulinate and a liquid base catalyst at lower temperatures (Table 1). No conversions were obtained. Valerolactone, methyl methacrylate, and acrolein were reacted with ethyl levulinate using hydrotalcite catalyst at 150° C. In the Parr reactor (Table 3). No conversions were obtained.

### Example 2.2.2

### Reactions with Acid Catalysts

Acid-catalyzed reactions of aldehydes were performed with several aldehydes. The reactions of ethyl levulinate with acetaldehyde and butyraldehyde in H₂SO₄ (Table 2) gave no conversion of the ethyl levulinate owing to rapid self-condensation of the aldehydes. In contrast, isobutyraldehyde gave a 63% conversion of ethyl levulinate with H₂SO₄ at 25° C.

The larger-scale reaction of isobutyraldehyde in the Parr reactor (Table 3) gave a 61% conversion of ethyl levulinate at 25° C. However, at 60° C., the reaction gave a 26% conversion.

### 2.3. Reactions of Levulinic Acid with Aldehydes

Levulinic acid can condense with aldehydes in acid or base conditions to give products similar to those obtained with ethyl levulinate. Some of these reactions were investigated in this project to determine whether feasible routes to higher-molecular-weight fuel additives can be found.

### Example 2.3.1

### Reactions with Base Catalysts

The reaction of levulinic acid with furfural in NaOH in ethanol at 60° C. did not result in any product (Table 1), nor did the reaction of levulinic acid with butyraldehyde in NaOH. However, the small-scale reaction of isobutyraldehyde in ethanolic NaOH resulted in 44% conversion of the levulinic acid. The reaction in the Parr reactor gave a 90% conversion of the levulinic acid.

### Example 2.3.2

### Reactions with Acid Catalysts

Levulinic acid was reacted with furfural in several acid catalysts. The reactions with a liquid system, PTSA (p-toluene sulphonicacid) in diglyme, gave good conversions of levulinic acid (Table 2). Reactions with a solid acid catalyst, Amberlite 15 (H+ form), also gave high conversions of levulinic acid, although the conversion decreased in solvent.

Reactions of levulinic acid with isobutyraldehyde in ethanolic H₂SO₄ gave a good conversion of the levulinic acid (Table 2), but acetaldehyde self-condensed. The reaction of larger amounts of isobutyraldehyde and levulinic acid in the Parr reactor without a solvent gave a lower conversion (30%). Isobutyraldehyde does undergo some self-condensation in strong acid, especially without a solvent present, which explains the lower conversion of levulinic acid.

### Discussion of Examples 2.1-2.2. Condensation of Ethyl Levulinate.

Subsequent acid or base-catalyzed condensation reactions of the ethyl levulinate produced in the decomposition of the cellulose or carbohydrate precursors (Example 1) result in its conversion to higher-molecular-weight species (1-7) that will be more appropriate for diesel and jet fuels or useful chemicals, such as plasticizers. Thus the 5-carbon acyl group of the ester is combined with aldehydes and ketones (Cₓ) to form (5+x)-carbon products. In addition, cycloparaffins are available from Diels-Alder reactions of the intermediates prepared from ethyl levulinate.

The general type of condensation reactions described here for aldehydes and levulinic acid or esters thereof are variations of the aldol condensation. When the aldol condensation occurs between a ketone at the carbon alpha to the carbonyl and an aldehyde, the reaction is called a Claisen-Schmidt condensation and between an aldehyde and a ketoester is a Stobbe condensation. Claisen-Schmidt condensation products from the reaction of levulinic acid and an aldehyde conducted with an acid or base catalyst typically are a mixture of the β-(or branched) and the δ-(or unbranched) forms and also include products from the condensation of levulinic acid or esters thereof with two or more aldehydes to give di- or polyadducts. Since an aldehyde with alpha hydrogens readily undergoes self-condensation, aldehydes without alpha hydrogens are preferred coreactants isobutyraldehyde can be condensed with a ketone because steric hinderance slows down the self-condensation. At least part of the ester products are hydrolyzed to carboxylic acids, depending on the catalyst.

The condensation reaction between furfural and ethyl levulinate in the presence of liquid base (e.g. dissolved) requires subsequent neutralization and destruction of the catalyst. And this generates a problematic alkaline wastewater stream. This problem is commonly solved by using solid base catalysts. A wide variety of solid bases have been examined for aldol condensation reactions. Examples include alkaline-earth oxides, K- and Li-promoted oxides, calcined hydrotalcites, zeolites, anion exchange resins, and polymer-supported guanidines.

Hydrotalcitelike layered double hydroxides (LDHs), also known as anionic clays, are natural or synthetic materials including positively charged brucitelike sheets with divalent and trivalent cations in the octahedral sites within the hydroxyl layers, plus an exchangeable interlayer anion. Carbonates are the interlayer anions in naturally occurring hydrotalcite. However, the number of counterbalancing ions is essentially unlimited, and LDHs intercalated by various simple inorganic, polyoxometalate, complex as welt as organic anions have been synthesized.

High catalytic performance can be achieved by thermal treatment of the LDHs, which transforms the hydrotalcite-like materials into mixed oxides. The mixed metal oxides are characterized by high specific surface areas, homogeneous dispersion of metals, and unique acid-base properties. The choice of suitable calcination conditions is a crucial factor influencing the features of the resulting oxides. The activation temperature must be high enough to decompose the interlayer anions, but it cannot exceed a critical temperature, at which the phase segregation and sintering effects take place. According to recent studies the appropriate temperature would be in the range 450°-550° C., and the calcined hydrotalcites show higher activity if they are calcined and stored in a N₂ atmosphere.

Hydrotalcites have recently received much attention as solid base catalysts. Their activated form is a potential solid base catalyst for a variety of organic transformations such as condensation, isomerization, anion exchangers, and epoxidation reactions. Numerous studies are reported on self-condensation of butanal, acetone, and cross-condensation reactions using hydrotalcites.

The Claisen-Schmidt or Stobbe condensation of ethyl levulinate with furfural can be effected with a liquid base system at lower temperatures (ambient to 60° C.), with removal of base catalyst from the products via neutralization and extraction. Solid base catalysts in the form of hydrotalcites are effective catalysts for the condensation of ethyl levulinate with furfural with temperatures of 135°-150° C. The products are a mix of mono- and difuryl-substituted levulinates. Much of the product is hydrolyzed to the acid form or is present as the lactone. Acid catalysts were not effective for the condensation of ethyl levulinate with furfural.

Isobutyraldehyde can be condensed with ethyl levulinate, but other aldehydes with an alpha hydrogen undergo self-condensation in competition with the cross-condensation desired. Like the furfural condensation, both liquid base and solid base systems will catalyze the isobutyraldehyde condensation, the solid base requiring a higher temperature. Good conversions were also obtained with liquid acid catalyst at low temperature, but not at high temperature. The products were a mixture of mono- and difuryl-substituted levulinate.

### Discussion of Examples 2.3—Condensation of Levulinic Acid.

The condensation reactions of levulinic acid obtained from the acid-catalyzed decompositions conducted in aqueous acid were successful, giving good conversions with furfural and isobutyraldehyde. Liquid acid catalysts in a solvent and solid acid catalysts without a solvent gave 68%-91% conversions when the temperature was over 60° C. Reactions of levulinic acid with furfural and isobutyraldehyde with a basic catalyst were not very successful, except for the reaction with isobutyraldehyde in excess of base. The reactions of levulinic acid with a solid base catalyst were not attempted.

### Example 2.4

### Solid Catalyst Optimization

Although the hydrotalcite catalysts were successful fur the ethyl levulinate condensation, further work was needed to optimize the reaction yields for aldol condensation of ethyl levulinate and furfural by developing various modifications of the hydrotalcite catalyst. Specifically, a series of hydrotalcite catalysts with varying composition were synthesized and characterized with respect to basic properties, and tests were performed to evaluate the catalytic activity of the hydrotalcite series.

In an effort to optimize the solid base composition, a number of solid base catalysts were prepared where the hydrotalcite composition and the method of preparation were changed. Twenty different calcined hydrotalcites were synthesized in amounts of 10 to 20 g in mesh size and 0.5-1.5 g in powder. The ratio of the Mg to Al was varied in several catalysts, additional elements (Sn, Zr, Ni, La) were added, a different base (urea) was added, pillaring organic acids (citric acid, edta) were added, and shear mixing was employed. Calcination temperature, aging time and temperature, and composition of hydrotalcites were varied in different samples. One sample was rehydrated.

Samples were synthesized according to following scheme:
1. Preparation of initial solutions: 2M NaOH/0.5M Na₂CO₃ and 2M (Mg+Al+other metals) salts solutions.
2. Addition of the solutions (dropwise) for 1-2 hours under vigorous stirring
3. Overnight aging at 60°-70° C. for 16 h
4. Filtration and washing with deionized H₂O until the pH is 7
5.Drying at 110° C. for 16 h
6. Calcination at 500° C. for 4 h
7. Division of the resulting solid into powder and mesh-sized part
8. Sealing of the mesh part under N₂

Mg was added as Mg(NO₃)₂, Al was added as Al(NO₃)₃; the materials can be added using any suitable salt known to one of skill in the art. Some of the samples were prepared in slightly different ways: some steps of the above scheme were changed, eliminated, or prolonged. Table 4 shows the synthesized samples and the conditions of the synthesis. “US mixing” indicates ultrasound mixing.

### Characterization

One of the most important parameters of the catalysts that affect the catalytic activity is the basicity of the catalyst. In order to characterize the different samples basicity Hammet indicators tests were run. All indicator solutions were prepared by dissolving the powdered compounds in methanol to form a 0.02M solution. Table 5 gives the characteristics of each indicator.

Every test was run in a test bottle: 1 ml of indicator solution was added to 9 ml of methanol and 20-50 mg of powdered hydrotalcite. Results of the test are presented in Table 6. One sample (HT30-450) was rehydrated by pumping N₂ saturated with H₂O through the compound. It was then used to see the difference in basicity of the usual and rehydrated hydrotalcite.

The Hammet indicator results show that most hydrotalcites have basic strength between 12.7 and 15, which is consistent with literature information. Some samples were found to have low or no basicity; one sample synthesized using shear mixing showed basicity higher than 15.

The rehydrated sample showed lower basicity than the initial hydrotalcite, which can be explained by the conversion of O₂ basic sites into weaker OH basic sites.

### Catalytic Activity Tests and Results

Two types of tests were used to determine catalytic activities of the synthesized catalysts:

Series 1: Batch Reaction
1. Hydrotalcite powder was placed into a pressure tube with a Teflon screw cap.
2. Approximately 5 mL of reagent solution was added, and the mass of all samples was always close to 0.5 g
3. The system was heated either in an oil bath or in an electric heating mantle for 4 hours.

Series 2: Continuous Reactor Reaction
1. 18-30-mesh-sized granules of a catalyst (10 g) were placed into a metal tube.
2. The tube with the loading was placed into a tube furnace.
3. The reagent solution was pumped through the tube at a constant speed of 1 mL/min but at increasing temperatures.
4. Effluent liquid product was collected and analyzed.

All collected solution samples were tested on a GC. For all the samples collected during continuous reactor reactions, diethyl ether was used as a diluent solvent for GC. Methyltetrahydrofuran was used for the samples from the batch reactions.

TABLE 4
Catalysts Prepared

| Name | Composition | Concentrations | Aging | Drying | Mass, g | Calcination | Mass, g |
|---|---|---|---|---|---|---|---|
| HT30-450 | Mg/Al = 3/1 | NaOH − 3.5M; (Mg + Al) − | 6 h, 75° C. | 16 h, 110° C. | 25 | 4 h, 450° C. | 13.8 |
| HT30-500 | | 2M | | | 24 | 4 h, 500° C. | 13.9 |
| HT30-550 | | | | | 25.2 | 4 h, 550° C. | 14.04 |
| HT30-US | | | US mixing | 16 h, 110° C. | 24.52 | 4 h, 500° C. | 13.65 |
| HT35 | Mg/Al = 3.5/1 | NaOH − 2M; (Mg + Al) − | 16 h, 75° C. | 16 h, 110° C. | 22 | 4 h, 500° C. | 14.11 |
| | | 2M | | | | | |
| HT25 | Mg/Al = 2.5/1 | NaOH − 2M; (Mg + Al) − | 16 h, 75° C. | 16 h, 110° C. | 23.7 | 4 h, 500° C. | 14.12 |
| | | 2M | | | | | |
| HT30-Sn | Mg/Al/Sn = 3/1/0.1 | NaOH − 2M; (Mg + Al + | 16 h, 80° C. | 16 h, 110° C. | 23.2 | 4 h, 500° C. | 13.44 |
| | | Sn) − 2M | | | | | |
| HT30-CA | Mg/Al = 3/1; Na₃C₆H₅O₇ | NaOH − 2M; Na₃C₆H₅O₇ − | 16 h, 90° C. | 16 h, 110° C. | 31.2 | 4 h, 500° C. | 17.6 |
| | used | 0.2M; (Mg + Al) − 2M | | | | | |
| HT30- | Mg/Al = 3/1; | NaOH − 2M; | 16 h, 60° C. | 18 h, 110° C. | 34.7 | 4 h, 500° C. | 21.1 |
| EDTA | Na₂H₂(EDTA) used | Na₂H₂(EDTA) − 0.15M; | | | | | |
| | | (Mg + Al) − 2M | | | | | |
| HT30-U | Mg/Al = 3/1; urea used, | (Mg + Al) = 0.15M; | None | 114 h (5 | 37.54 | 4 h, 500° C. | 6 |
| | no base | (Mg + Al)/urea = 4; 90° C., | | days), 110° C. | | | |
| | | 20 h | | 18 h, 110° C. | | | |
| HT30-Sh | Mg/Al = 3/1 | NaOH − 2M; (Mg + Al) − | 16 h, 50° C. | 16 h, 110° C. | 17.1 | 4 h, 500° C. | 9.91 |
| | | 2M; shear mixing | | | | | |
| HT40-Zr | Mg/Al = 4/1; 0.25 Zr⁴⁺ | NaOH − 2M; (Mg + Al + | 16 h, 65° C. | 16 h, 110° C. | 18.34 | 4 h, 500° C. | 11.63 |
| | used | Zr) − 2M | | | | | |
| HT26-Ni | Mg/Al = 2.6/1; 0.4 Ni²⁺ | NaOH − 2M; (Mg + Al + | 16 h, 65° C. | 16 h, 110° C. | 22.3 | 4 h, 500° C. | 13.4 |
| | used; (Mg + Ni)/Al = 3/1 | Ni) − 2M | | | | | |
| HT40-Sn | Mg/Al = 4/1; 0.25 Sn⁴⁺ | NaOH − 2M; (Mg + Al + | 16 h, 65° C. | 16 h, 110° C. | 22 | 4 h, 500° C. | 13.92 |
| | used; Mg/(Sn + Al) = 3/1 | Sn) − 2M | | | | | |
| HT31-Sn² | Mg/Al = 3.1/1; 0.25 Sn²⁺ | NaOH − 2M; (Mg + Al + | 16 h, 65° C. | 16 h, 110° C. | 23.9 | 4 h, 500° C. | 11.93 |
| | used; (Mg + Sn²)/(Al + | Sn) − 2M | | (small bowl) | | | |
| | Sn³) = 3/1 | | | | | | |
| HT40-Ti | Mg/Al = 4/1; 0.25 Ti⁴⁺ | NaOH − 2M; (Mg + Al + | 16 h, 65° C. | 16 h, 110° C. | 24.04 | 4 h, 500° C. | 13.96 |
| | used; Mg/(Ti + Al) = 3/1 | Ti) − 2M | | | | | |
| HT30-HA | Mg/Al = 3/1; humic acid | NaOH − 2M; (Mg + Al) − | 16 h, 65° C. | 16 h, 110° C. | 33.59 | 4 h, 500° C. | 20.17 |
| | sodium salt used, no | 2M | | | | | |
| | sodium carbonate | | | | | | |
| HT40-La | Mg/Al = 4/1; 0.25 La³+ | NaOH − 2M; (Mg + Al + | 16 h, 65° C. | 16 h, 110° c. | 23.75 | 4 h, 500° C. | 13.94 |
| | used; Mg/(Al + La) = 3/1 | La) − 2M | | | | | |
| HT30-2Sh | Mg/Al = 3/1 | NaOH − 2M; (Mg + Al) − | No aging | 16 h, 110° C. | 24 | 4 h, 500° C. | 14.23 |
| | | 2M; shear mixing | | | | | |

TABLE 5
Indicator test pH characteristics

| Name | pH range of color change | Symbol |
|---|---|---|
| Phenolphtalein | >10 | Ph |
| Thymolphtalein | >10.5 | Th |
| Tropaeolin O | >12.7 | Tr |
| Dinitroaniline | >15 | D |

TABLE 6
Hammett basicity results

| Sample | Ph | Th | Tr | D |
|---|---|---|---|---|
| HT30-US-500 | + | + | + | − |
| HT26-Ni-500 | + | + | + | − |
| HT30-550 | + | + | + | − |
| HT40-Sn-500 | + | + | + | − |
| HT30-Sn-500 | + | + | + | − |
| HT30-HA-500 | + | ? | + | − |
| HT40-La-500 | + | + | + | − |
| HT30-Sh-500 | + | + | + | + |
| HT25-500 | + | −+¹ | − | − |
| HT35-500 | + | + | + | − |
| HT30-500 | + | + | + | − |
| HT40-Ti-500 | + | + | + | − |
| HT30-CA-500 | + | + | + | − |
| HT40-Zr-500 | + | + | + | − |
| HT31-Sn(2)-500 | + | + | + | − |
| HT30-Edta-500 | + | + | + | − |
| HT30-450 | + | + | + | − |
| HT30-U-500 | ND² | +−³ | +− | ND² |
| HT30-450 (rehydrated) | ND² | −+¹ | − | ND² |

¹Almost no color change.
²Not determined.
³Some color change but much less than with other samples.

### Results for Batch Reaction Tests

Table 7a and 7b shows the conversion results and ⁻the conditions of the batch reactions (Series 1). For starting materials, about 0.64 g of furfural, and about 0.96 g ethyl levulinate, were used. HT-sh indicates hydrotalcite that was prepared using shear mixing (blender). Good conversions of ethyl levulinate were obtained for several of the catalysts. The HT30-500 catalyst gave a 66% conversion of the levulinate in 4 hours at 165° C. The similar composition prepared with citrate addition was also fairly reactive. Some catalysts with other elements added had little or no activity. It is likely these may have increased acidic sites, but at the expense of losing basic sites.

TABLE 7a
Conditions and Conversions for Batch Reactions

| | | | | Average | | Ret. | | Ret. | | Ret. | | | |
|---|---|---|---|---|---|---|---|---|---|---|---|---|---|
| | | | | Temp., | Highest, | Time, | A¹ | Time, h | A | Time, h | A | | |
| Sample | Solvent | Mass | Time, h | ° C. | ° C. | h (F) | (F)² | (DG)³ | (DG) | (EL)⁴ | (EL) | M⁵ (F) | M (EL) |
| HT30-500-p | Ether | 0.5 | 3 | 130 | 140 | 3.4 | 205 | 4.4 | 206 | 8 | 180 | 0.69 | 0.74 |
| HT30-450 US | Ether | 0.5 | 1 | Not controlled | | 3.4 | 1273 | 4.4 | 1365 | 8.1 | 1737 | 0.65 | 1.08 |
| HT30-450 | Ether | 0.5 | 3 | 130 | 140 | 3.4 | 55 | 4.4 | 108 | 8 | 117 | 0.35 | 0.92 |
| HT30-450 | MeTHF | 0.5 | 19 | 140 | 150 | 3.4 | 60 | 4.4 | 1669 | 8 | 659 | 0.02 | 0.34 |
| HT30-450 | MeTHF | 0.5 | 4 | 145 | 160 | 3.4 | 108 | 4.4 | 159 | 8 | 114 | 0.43 | 0.62 |
| HT30-500 | MeTHF | 0.5 | 4 | 165 | 180 | 3.5 | 29 | 4.4 | 437 | 8 | 170 | 0.04 | 0.34 |
| HT30-500 | MeTHF | 0.5 | 4 | 150 | 160 | 3.4 | 168 | 4.4 | 446 | 9 | 276 | 0.24 | 0.54 |
| HT30-CA | MeTHF | 0.5 | 4 | 155 | 165 | 3.4 | 77 | 4.4 | 310 | 8 | 208 | 0.16 | 0.58 |
| | | | | | | 3.4 | 90 | 4.4 | 277 | 8 | 250 | 0.21 | 0.79 |
| | | | | | | 3.4 | 103 | 4.4 | 336 | 8 | 239 | 0.20 | 0.62 |
| HT30-Sn | MeTHF | 0.52 | 4 | 150 | 155 | 3.4 | 1443 | 4.4 | 1585 | 8.1 | 1656 | 0.58 | 0.91 |
| | | | | | | 3.4 | 317 | 4.4 | 348 | 8 | 345 | 0.58 | 0.86 |
| HT26-Ni | MeTHF | 0.44 | 4 | 155 | 180 | 3.4 | 125 | 4.4 | 137 | 8 | 146 | 0.58 | 0.93 |
| | | | | | | 3.4 | 890 | 4.4 | 932 | 8 | 876 | 0.61 | 0.82 |
| HT40-Zr | MeTHF | 0.51 | 4 | 153 | 158 | 3.4 | 271 | 4.4 | 283 | 8 | 285 | 0.61 | 0.88 |
| | | | | | | 3.4 | 305 | 4.4 | 317 | 8 | 319 | 0.62 | 0.88 |
| HT31-Sn(2) | MeTHF | 0.51 | 4 | 150 | 155 | 3.4 | 359 | 4.4 | 361 | 8 | 334 | 0.64 | 0.80 |
| | | | | | | 3.4 | 326 | 4.4 | 322 | 8 | 367 | 0.65 | 0.99 |
| HT30-Sh | MeTHF | 0.5 | 4 | 155 | 190 | 3.4 | 163 | 4.4 | 213 | 8 | 242 | 0.49 | 0.99 |
| | | | | | | 3.4 | 102 | 4.4 | 133 | 8 | 165 | 0.49 | 1.08 |
| HT40-La | MeTHF | 0.5 | 4 | 150 | 165 | 3.4 | 168 | 4.4 | 168 | 8 | 200 | 0.64 | 1.04 |
| | | | | | | 3.5 | 208 | 4.5 | 216 | 8.1 | 247 | 0.62 | 0.99 |
| HT30-450 | MeTHF | 0.49 | 4 | 150 | 155 | 3.4 | 452 | 4.4 | 445 | 8.1 | 416 | 0.65 | 0.81 |

¹Area under the peak from GC analyzer. Ret. time in this table corresponds to the retention time of the material in the instrument.
²Furfural (F).
³Diglyme solvent (DG).
⁴Ethyl levulinate (EL).
⁵Amount (mass) remaining.

TABLE 7b
Conditions and Conversions for Hydrotalcite Batch Reactions
(~0.5 g catalyst/5 mL reagents)

| | | | Temp. | Conversion | Conversion |
|---|---|---|---|---|---|
| Sample | Solvent | Time, h | ° C. | (F) % | (EL) % |
| HT30-500-p | Ether | 3 | 130 | 0 | 23 |
| HT30-450 US | Ether | 1 | — | 0 | 0 |
| HT30-450 | Ether | 3 | 130 | 45 | 4 |
| HT30-450 | MeTHF | 19 | 140 | 97 | 65 |
| HT30-450 | MeTHF | 4 | 145 | 33 | 35 |
| HT30-500 | MeTHF | 4 | 165 | 94 | 65 |
| HT30-500 | MeTHF | 4 | 150 | 63 | 44 |
| HT30-CA | MeTHF | 4 | 155 | 75 | 40 |
| | | | | 67 | 18 |
| | | | | 69 | 35 |
| HT30-Sn | MeTHF | 4 | 150 | 9 | 5 |
| | | | | 9 | 10 |
| HT26-Ni | MeTHF | 4 | 155 | 9 | 3 |
| | | | | 5 | 15 |
| HT40-Zr | MeTHF | 4 | 153 | 5 | 8 |
| | | | | 3 | 8 |
| HT31-Sn(2) | MeTHF | 4 | 150 | 0 | 17 |
| | | | | 0 | 0 |
| HT30-Sh | MeTHF | 4 | 155 | 23 | 0 |
| | | | | 23 | 0 |
| HT40-La | MeTHF | 4 | 150 | 0 | 0 |
| | | | | 3 | 0 |
| HT30-450 | MeTHF | 4 | 150 | 0 | 16 |

### Results for Continuous Reactor Tests

Three of the solid base catalysts were tested in a bed configuration in a continuous tube reactor with a mixture of ethyl levulinate and furfural diglyme pumped through the tube at 1 mL/min.

### HT30-500 Test

A bed was prepared with the 10 g of HT30-500 catalyst. The reaction temperature started at 60° C. and was increased to 160° C. over a period of about 6 hours. The HT30-500 catalyst gave no conversion below 150° C., but at 160° C., 47% of the ethyl levulinate was reacted. More of the furfural reacted, owing to reaction of two furfurals with one levulinate. This is consistent with the results from the batch tests performed with this catalyst.

### HT35-500 Test

A similar experiment was performed with a bed of the HT35-500 with Mg/Al=3.5. This test showed no reaction below 160° C. and gave about 30% conversion of ethyl levulinate at 160° C. The higher Mg/Al ratio was intended to produce greater basicity. However, these results with the catalysts with the larger amount of magnesium and the earlier results with the commercial catalyst (Mg/Al=2) show that optimum conversions are obtained with a catalyst composition with lower Mg/Al ratios.

### HT40-SN500

The HT with added Sn gave no reaction up to 180° C. After 1 hour at 180° C., the reaction was discontinued.

### Discussion of Example 2.4

The basic properties of the catalysts were determined by reactions with indicator solutions. Most exhibited a pH of >12.7 and <15, and there was little pH distinction between the catalysts.

Batch testing of the catalysts for e levulinate and furfural condensation was conducted in a small heat-jacketed stirred reaction vessel at 130° to 165° C. The conversion of ethyl levulinate was measured as the test of catalyst reactivity. The composition with the Mg/Al ratio=3 (HT30-500) gave the highest conversions (66%). This was slightly less than that achieved with the commercial catalyst with Mg/Al=2. Addition of other elements to the composition resulted in inactive catalysts.

Three of the solid base catalysts were tested in a bed configuration in a continuous tube reactor with a mixture of ethyl levulinate and furfural diglyme pumped through the tube at 1 mL/min. The reaction temperature started at 60° C. and was increased to 160° C. The HT30-500 catalyst gave no conversion below 150° C., but at 160° C., 47% of the ethyl levulinate was reacted. The HT35-500 with Mg/Al=3.5 gave about 30% conversion at 160° C. The HT with added Sn gave no reaction up to 180° C. These results show that optimum conversions are obtained with a catalyst composition with tower Mg/Al ratios.

### Example 2.5

### Analysis of Condensation Products

The results of analysis of a typical condensation product sample are given in Table 8 as determined by gas chromatography-mass spectroscopy. (GC-MS) and retention times. The components include the original ethyl levulinate ester, some levulinic acid, ethanol, the two isomers of the monocondensation product, the biscondensation product, several thermal decomposition products of the condensation products believed to be produced as an artifact in the GC-MS inlet during the analysis, and the solvent MeTHF. The sample likely contained a considerable amount of acidic condensation products as described above, which decompose in the GC inlet to give the decomposition products listed in the analysis (Table 8) as well as others that did not show up in the analysis.

TABLE 8
Starting Composition for Hydrotreating at Pacific Northwest National
Laboratory (PNNL).

| Peak | Ret Time, h | M/e | Name |
|---|---|---|---|
| 1 | 4.43 | 86 | 2-methylTHF |
| 2 | 4.78 | 102 | Unknown ester |
| 3 | 7.36 | | Unknown decomp. prod. |
| 4 | 7.47 | 150 | Unknown decomp. prod. |
| 5 | 8.12 | 116 | Levulinic acid |
| 6 | 8.32 | 144 | Ethyl Levulinate |
| 7 | 12.8 | 222 | Ethyl 3-furfurylidene-4-oxo-pentanoate |
| | | | (monocond. prod.) |
| 8 | 13.5 | 222 | Ethyl 6-furfuryl-4-oxo-5-hexenoate |
| | | | (monocond. prod.) |
| 9 | 13.6 | 216 | 1,5-difurfuryl-1-penten-3-one |
| | | | (decomp. prod.) |
| 10 | 16.74 | 300 | Ethyl 3-fufurylidene-6-furfuryl-4-oxo-5- |
| | | | hexenoate (biscond.prod.) |

### Example 3.1

### Catalytic Hydrogenation

In the levulinate biorefinery art, a catalytic hydrogenation is performed on the ketoacid and ketoester intermediates mixture produced in the condensation unit. These oxygen functional groups are reduced along with unsaturation, resulting in the formation of the mixtures of paraffins, isoparaffins, cycloparaffins and alkylaromatics. Under milder conditions, a tetrahydrofuran ring forms and the existing furan ring is reduced to a tetrahydrofuran. The substituted tetrahydrofurans are utilized as a high-cetane diesel fuel additive.

Hydrotreating examples utilized a composite of several of the batch reactor products produced in the condensation examples (Example 2). Products were analyzed by GC to determine conversions of the fed and by GC-MS to elucidate the structures of the product components. In the first series of tests conducted in a multiparallel microflow reactor block with a variety of catalysts at 200° C. (Table 9), most of the catalysts gave good conversions of the condensate product components as well as the ethyl levulinate remaining in the feed. Conversion data for the hydrotreating runs are shown in FIG. 2.

TABLE 9
Catalysts Used in PNNL Hydrotreating Runs

| | |
|---|---|
| 5.0% Ru on Carbon (Hyperion) | 14388-79-4 |
| 2.5% Pd/2.2% Re on Carbon (Norit ROX 0.8) | 58419-10-1 |
| 5.0% Re/3.0% Ru on Carbon (Hyperion) | 14388-87-2 |
| 5.0% Re/2.0% Pt on Carbon (Norit ROX 0.8) | 14388-87-1 |
| 5.0% Re/5.0% Ir on Carbon (Norit ROX 0.8) | 14388-87-5 |
| 5.0% Fe/1.0% Pt on Carbon (Norit ROX 0.8) | 58959-136-7 |
| 5.0% Os/1.0% Rh on Carbon (Norit ROX 0.8) | 58959-128-2 |
| 5.0% Rh on Alumina (Puralox) | 14388-39-1 |
| 5.0% Ni/1.0% Re on Carbon (Norit ROX 0.8) | 102654-A2 |
| 5.0% Re on Carbon (Norit ROX 0.8) | 14388-93-2 |

A sample of the hydrotreated products was shipped back to the EERC to determine the products. Analysis of the hydrotreating products gave similar products in various yields. The results from the GC-MS of one of the samples is shown in Table 11. From the molecular weights and fragmentation pattern observed, a large number of component products were identified or partially identified owing to lack of standards.

Several peaks included the starting materials and solvent. The large number of products were formed because of the complexity of the starting feed as well as partial hydrogenation. For example, in some products, the furan ring was present intact or unchanged; in other products hydrogenation produced tetrahydrofurans. Both esters and intones were present. The acid products were likely decomposed in the inlet as in the case of starting feed components.

TABLE 11
Components in Hydrotreating Product with PNNL catalyst.

| | Ret. | | |
|---|---|---|---|
| Peak | Time, h | M/e | Name |
| 1 | 12.54 | 198 | 4-tetrahydrfurfuryl-3-aceto-4-hydroxypentanoic |
| 2 | 12.92 | | lactone THF derivative |
| 3 | 13.01 | 182 | THF derivative |
| 5. | 13.16 | 228 | Ethyl 6-tetrahydrofurfuryl-4-oxo-5-hexenoate |
| 6 | 13.95 | 232 | BisTHF decomp product |
| 7 | 14.98 | 304 | Ethyl 3-fufurylidene-6-furfuryl-4-hydroxy-5- |
| | | | hexanoate |
| 8 | 15.47 | 308 | Ethyl 3-fufurylidene-6-tetrahydrofurfuryl-4- |
| | | | hydroxy-5-hexanoate |
| 9 | 15.69 | 310 | Ethyl 3-tetrahydrofufurylidene-6- |
| | | | tetrahydrofurfuryl-4-oxo-5-hexanoate |
| 10 | 15.74 | 304 | Bis furanyl derivative. |
| 11. | 15.99 | 312 | Ethyl 3-tetrahydrofufurylidene-6- |
| | | | tetrahydrofurfuryl-4-hydroxy-5-hexanoate |
| 12 | 16.03 | 312 | Ethyl 3-tetrahydrofufurylidene-6- |
| | | | tetrahydrofurfuryl-4-hydroxy-5-hexanoate |
| | | | (stereoisomer of 11) |

### Discussion of Example 3.1

### Hydrotreating Condensation Products

Most of the catalysts employed for hydrotreating the condensation products gave reduction products, although many of the products were only partially hydrogenated. Ruthenium, rhodium, palladium, platinum, and even iron and nickel gave good conversions. Rhenium was included in the compositions to achieve better reduction of the oxygens, but under the conditions used, few if any of the oxygens were reduced off. Rhenium by itself was not effective for reduction.

### Example 3.2

### Hydrotreating Flow-Through Reactor with a Catalyst Bed of Cu-Modified Pd Catalyst over Carbon

The feed for the hydrotreating was obtained from the ethyl levulinate and furfural condensation. The feed was distilled at 160° C. under house vacuum prior to use to remove MeTHF and furfural.

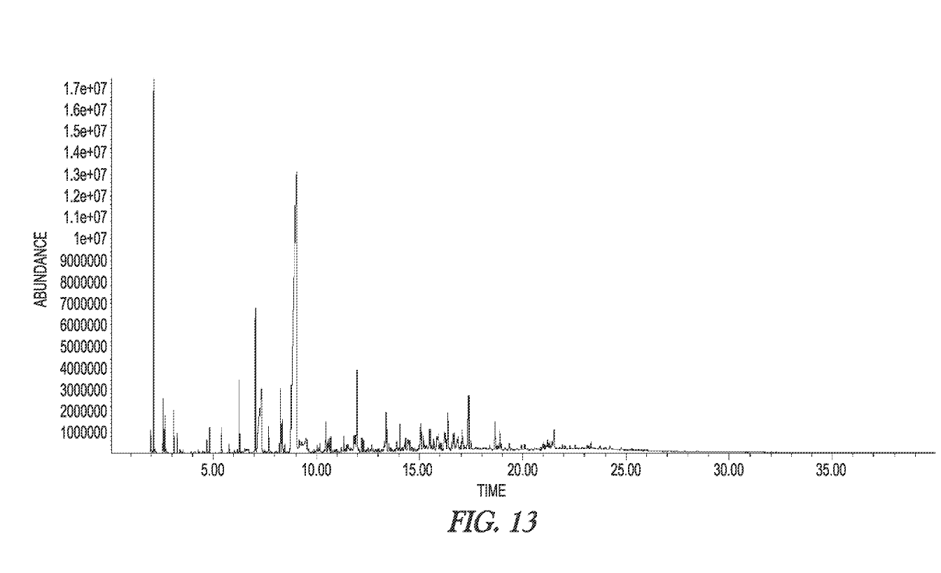

A small column was packed with 24 mL of Cu/Pd/carbon catalyst, and the furfural-free condensation product was pumped through the catalyst bed at 300° C. and 1000 psi (200 sccm hydrogen) at a liquid hourly space velocity (LHSV) of 0.5 hr⁻¹. The product was collected and analyzed by GC-MS. FIG. 13 shows the GC-MS spectrum of the product. A large number of components were present. A selected ion chromatogram for the m/e=71 ion showed that many of the components had a significant m/e=71 ion, corresponding to the tetrahydrofurfuryl structure. Several of the peaks were ethyl esters, but lactones also appeared to be present. Very few hydrocarbons were present.

### Example 3.3

### Hydrotreating Flow-Through Reactor with Sulfided Ni—Mo Catalyst

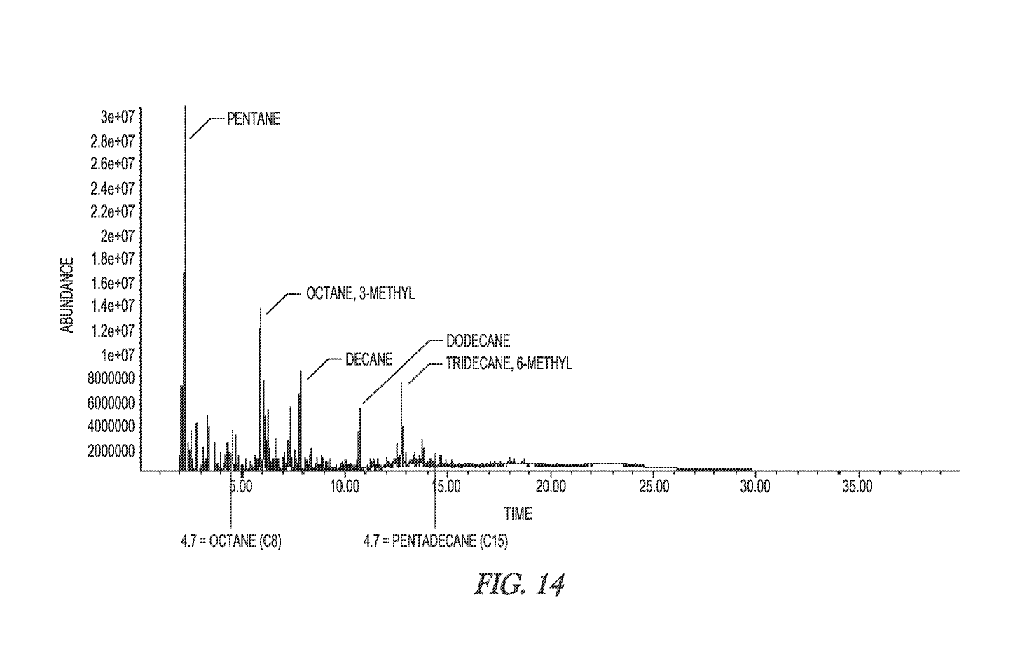

Feed 1: Prehydrogenated Feed. The feed for the first hydrotreating with the Ni—Mo catalyst was obtained from the prior (partial) hydrotreating with the Cu/Pd/carbon catalyst The furfural and MeTHF were removed. A small column was packed with 24 of Ni—Mo catalyst and sulfided with dimethylsuifide in dodecane. The furfural-free condensation product was pumped through the catalyst bed at 300° C. and 1000 psi (200 sccm hydrogen) at a LHSV of 0.5 hr⁻¹. The product was collected and analyzed by GC-MS. FIG. 14 shows the GC-MS spectrum of the product. A large number of hydrocarbon components were present, mostly in the range of C9-C15, and predominantly 3-methyloctane, nonane, 3-ethyloctane, and decane. Some pentane was produced via hydrogenation of the residual of ethyl levulinate present in the feed.

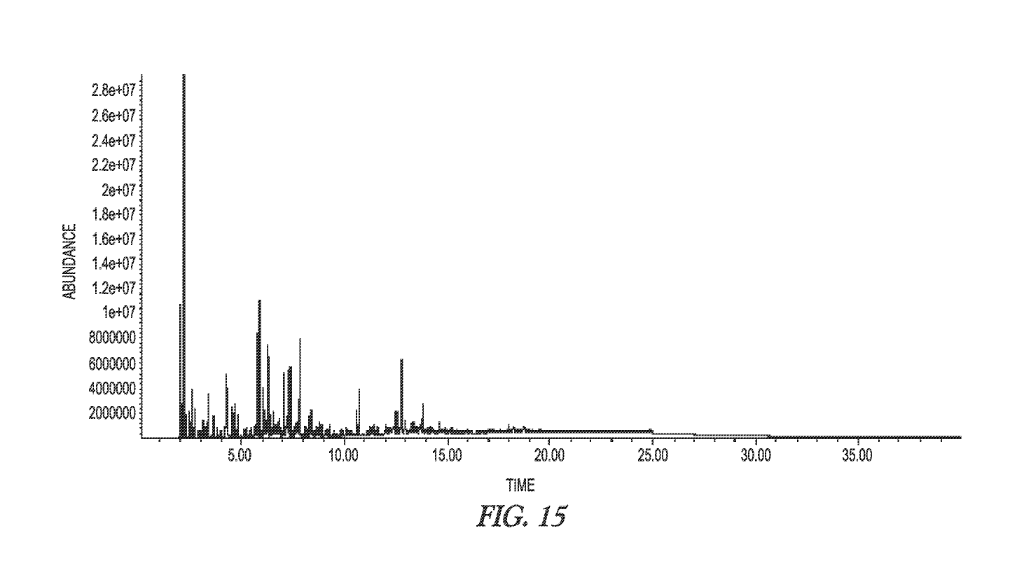

Feed 2: Fed from Condensation Reaction. Feed 2 for the hydrotreating with the Ni—Mo catalyst was obtained from the ethyl levulinate and furfural condensation. A small column was packed with 24 mL of Ni—Mo catalyst and sulfided with dimethylsulfide in dodecane. The furfural-free condensation product was pumped through the catalyst bed at 300° C. and 1000 psi (200 sccm hydrogen) at a LHSV of 0.5 hr⁻¹. The product was collected and analyzed by GC-MS. FIG. 15 shows the GC-MS spectrum of the product. A large number of hydrocarbon components were present, mostly in the range of C9-C15.

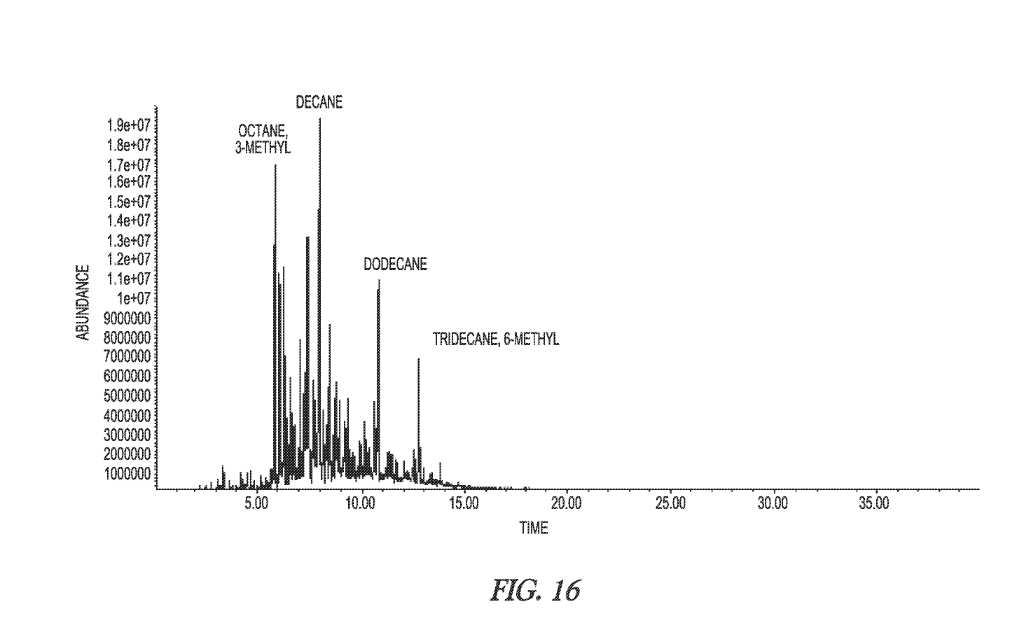

Distillation of Ni—Mo-hydrotreated product from Feed 2. Distillation produced a center cut with the characteristics listed in Table 12. The GC-MS chromatogram shown in FIG. 16 shows midrange alkanes.

TABLE 12
Characteristics of Center Cut

| Sample | Freeze Point, ° C. | Flash Point, ° C. | Density, g/mL |
|---|---|---|---|
| Jet Fraction | −48.4 | 36.0 | 0.76 |
| JP-8 Spec | <−47 | >38 | 0.775-0.840 |

The terms and expressions which have been employed are used as terms of description and not of limitation, and there is no intention that in the use of such terms and expressions of excluding any equivalents of the features shown and described or portions thereof, but it is recognized that various modifications are possible within the scope of the invention claimed. Thus, it should be understood that although the present invention has been specifically disclosed by preferred embodiments and optional features, modification and variation of the concepts herein disclosed may be resorted to by those skilled in the art, and that such modifications and variations are considered to be within the scope of this invention as defined by the appended claims.

### Additional Embodiments

The present invention provides for the following exemplary embodiments, the numbering of which is not to be construed as designating levels of importance:

Embodiment 1 provides a method for converting a source of C6 sugar into a mixture of hydrotreated compounds including thermocatalytically reacting a source of C6 sugar to produce a solution including at least one of levulinic acid and levulinic acid ester; extracting at least one of the levulinic acid and the levulinic acid ester from the solution using a cyclic ether; condensing at least a portion of at least one of the levulinic acid and the levulinic acid ester with at least one of C4-C11 aldehydes, C4-C11 ketones, C4-C11 esters, or C4-C11 ketoacids to produce a condensation product; and hydrotreating at least a portion of the condensation product to provide a mixture of hydrotreated compounds.

Embodiment 2 provides the method of Embodiment 1, wherein the source of C6 sugar includes at least one of cellulosic material and starch material.

Embodiment 3 provides the method of any one of Embodiments 1-2, wherein the source of C6 sugar includes wood, wood pulp, pulping sludge, particleboard, paper, grass, or an agricultural by-product.

Embodiment 4 provides the method of any one of Embodiments 1-3, wherein the source of C6 sugar includes an agricultural by-product including at least one of straw, stalks, cobs, beets, beet pulp, seed hulls, bagasse, algae, corn starch, potato waste, sugar cane, and fruit waste.

Embodiment 5 provides the method of any one of Embodiments 1-4, wherein the thermocatalytic reaction is conducted with acid in at least one of water and alcohol.

Embodiment 6 provides the method of any one of Embodiments 1-5, wherein thermocatalytically reacting includes depolymerizing the source of C6 sugar in a thermal unit to provide a soluble carbohydrate intermediate prior to reacting catalytically to produce at least one of the levulinic acid and the levulinic acid ester.

Embodiment 7 provides the method of Embodiment 6, wherein the soluble carbohydrate intermediate includes anhydrosugar.

Embodiment 8 provides the method of any one of Embodiments 6-7, wherein thermocatalytic reaction of the anhydrosugar is catalyzed by a solid acid catalyst.

Embodiment 9 provides the method of any one of Embodiments 1-8, wherein the C4-C11 aldehyde is branched or aromatic.

Embodiment 10 provides the method of Embodiment 9, wherein the C4-C11 aldehyde is selected from the group consisting of isobutyraldehyde, furfural, hydroxymethylfurfural, substituted benzaldehydes, and cyclic aliphatic aldehydes.

Embodiment 11 provides the method of Embodiment 10, wherein the furfural is prepared from a source of C5 sugars.

Embodiment 12 provides the method of any one of Embodiments 1-11, wherein the C4-C11 ketone is selected from the group consisting of 1,2 diketones, 1,2 ketoesters, 1,4 ketoesters, 1,4 ketoacids, 2,3-butanedione, and 2,3-pentanedione.

Embodiment 13 provides the method of any one of Embodiments 1-12, wherein the condensing includes condensing in the presence of a catalyst.

Embodiment 14 provides the method of Embodiment 13, wherein the catalyst includes a solid base catalyst.

Embodiment 15 provides the method of any one of Embodiments 13-14, wherein the catalyst includes hydrotalcite or impregnated hydrotalcite.

Embodiment 16 provides the method of any one of Embodiments 13-15, wherein the catalyst is a solid acid catalyst

Embodiment 17 provides the method of any one of Embodiments 13-17, wherein the catalyst is a free radial initiator including manganese(III) acetate.

Embodiment 18 provides the method of any one of Embodiments 13-17, wherein the solid acid catalyst for the condensation includes a transition metal or a heterogeneous catalyst including at least one of sulfated titania, sulfated zirconia, sulfated alumina, sulfonated activated carbon, sulfonated mesoporous carbon, sulfonated carbon composite, and sulfonated polymer.

Embodiment 19 provides the method of any one of Embodiments 1-18, further including separating the mixture of hydrotreated compounds to give at least one of a fuel or fuel blendstock.

Embodiment 20 provides the method of any one of Embodiments 1-19, wherein the hydrotreating of the condensation products includes producing cyclic ethers.

Embodiment 21 provides the method of Embodiment 20, further including hydrotreating the cyclic ethers to produce one of diesel, diesel blendstock, jet fuel, jet fuel blendstock, and any combination thereof.

Embodiment 22 provides the method of Embodiment 21, further including coprocessing the cyclic ethers with at least one of free fatty acids, natural oils, tall oils, BTX (benzene, toluene, and xylenes), condensation products derived from BTX, and any combination thereof.

Embodiment 23 provides the method of any one of Embodiments 21-22, wherein the condensation product is separated to a mixture including materials having a chain length of about C10-C15, including n-alkanes, isoalkanes, cycloalkanes, and arylalkanes.

Embodiment 24 provides the method of any one of Embodiments 1-23, wherein at least a portion of an organic liquid is separated from an intermediate or final product of the method for use as solvent.

Embodiment 25 provides the method of any one of Embodiments 1-24, wherein the cyclic ether used to extract at least one of the levulinic acid and the levulinic acid ester is methyl tetrahydrofuran.

Embodiment 26 provides the method of Embodiment 24, wherein the methyl tetrahydrofuran at least partially includes methyl tetrahydrofuran separated from an intermediate or final product mixture of the method.

Embodiment 27 provides the method of any one of Embodiments 1-26, further including, partial evaporation of water prior to the extracting of at least one of levulinic acid and levulinic acid ester from the solution including at least one of levulinic acid and levulinic acid ester.

Embodiment 28 provides the method of any one of Embodiments 1-27, further including the hydrodeoxygenation C5 oxygen containing carbon compounds to form gasoline.

Embodiment 29 provides the apparatus or method of any one or any combination of Embodiments 1-28 optionally configured such that all elements or options recited are available to use or select from.

1. A method for converting a source of C6 sugar into a mixture of hydrotreated compounds comprising:
thermocatalytically reacting a source of C6 sugar to produce a solution comprising at least one of levulinic acid and levulinic acid ester;
extracting at least one of the levulinic acid and the levulinic acid ester from the solution using a cyclic ether;
condensing at least a portion of at least one of the levulinic acid and the levulinic acid ester with at least one of C4-C11 aldehydes, C4-C11 ketones, C4-C11 esters, or C4-C11 ketoacids to produce a condensation product; and
hydrotreating at least a portion of the condensation product to provide a mixture of hydrotreated compounds.

2. The method of claim 1, wherein the source of C6 sugar comprises at least one of cellulosic material and starch material.

3. The method of claim 1, wherein the source of C6 sugar comprises wood, wood pulp, pulping sludge, particleboard, paper, grass, or an agricultural by-product.

4. The method of claim 1, wherein the source of C6 sugar comprises an agricultural by-product comprising at least one of straw, stalks, cobs, beets, beet pulp, seed hulls, bagasse, algae, corn starch, potato waste, sugar cane, and fruit waste.

5. The method of claim 1, wherein the thermocatalytic reaction is conducted with acid in at least one of water and alcohol.

6. The method of claim 1, wherein thermocatalytically reacting comprises depolymerizing the source of C6 sugar in a thermal unit to provide a soluble carbohydrate intermediate prior to reacting catalytically to produce at least one of the levulinic acid and the levulinic acid ester.

7. The method of claim 6, wherein the soluble carbohydrate intermediate comprises anhydrosugar.

8. The method of claim 7, wherein the thermocatalytic reaction of the anhydrosugar is catalyzed by a solid acid catalyst.

9. The method of claim 1, wherein the C4-C11 aldehyde is branched or aromatic.

10. The method of claim 9, wherein the C4-C11 aldehyde is selected from the group consisting of isobutyraldehyde, furfural, hydroxymethylfurfural, substituted benzaldehydes, and cyclic aliphatic aldehydes.

11. The method of claim 10, wherein the furfural is prepared from a source of C5 sugars.

12. The method of claim 1, wherein the C4-C11 ketone is selected from the group consisting of 1,2 diketones, 1,2 ketoesters, 1,4 ketoesters, 1,4 ketoacids, 2,3-butanedione, and 2,3-pentanedione.

13. The method of claim 1, wherein the condensing comprises condensing in the presence of a catalyst.

14. The method of claim 13, wherein the catalyst comprises a solid base catalyst.

15. The method of claim 13, wherein the catalyst comprises hydrotalcite or impregnated hydrotalcite.

16. The method of claim 13, wherein the catalyst is a solid acid catalyst

17. The method of claim 13, wherein the catalyst is a free radial initiator comprising manganese(III) acetate.

18. The method of claim 13, wherein the solid acid catalyst for the condensation comprises a transition metal or a heterogeneous catalyst comprising at least one of sulfated titania, sulfated zirconia, sulfated alumina, sulfonated activated carbon, sulfonated mesoporous carbon, sulfonated carbon composite, and sulfonated polymer.

19. The method of claim 1, further comprising separating the mixture of hydrotreated compounds to give at least one of a fuel or fuel blendstock.

20. The method of claim 1, wherein the hydrotreating of the condensation products comprises producing cyclic ethers.

21. The method of claim 20, further comprising hydrotreating the cyclic ethers to produce one of diesel, diesel blendstock, jet fuel, jet fuel blendstock, and any combination thereof.

22. The method of claim 21, further comprising coprocessing the cyclic ethers with at least one of free fatty acids, natural oils, tall oils, BTX (benzene, toluene, and xylenes), condensation products derived from BTX, and any combination thereof.

23. The method of claim 21, wherein the condensation product is separated to a mixture including materials having a chain length of about C10-C15, comprising n-alkanes, isoalkanes, cycloalkanes, and arylalkanes.

24. The method of claim 1, wherein at least a portion of an organic liquid is separated from an intermediate or final product of the method for use as solvent.

25. The method of claim 1, wherein the cyclic ether used to extract at least one of the levulinic acid and the levulinic acid ester is methyl tetrahydrofuran.

26. The method of claim 24, wherein the methyl tetrahydrofuran at least partially comprises methyl tetrahydrofuran separated from an intermediate or final product mixture of the method.

27. The method of claim 1, further comprising, partial evaporation of water prior to the extracting of at least one of levulinic acid and levulinic acid ester from the solution comprising at least one of levulinic acid and levulinic acid ester.

28. The method of claim 1, further comprising the hydrodeoxygenation of C5 oxygen containing carbon compounds to form gasoline.
